(19) United States
(12) Patent Application Publication
(10) Pub. No.: US 2017/0042104 A1
(43) Pub. Date: Feb. 16, 2017

(54) SUGARCANE PROCESS

(71) Applicant: E I DU PONT DE NEMOURS AND COMPANY, Wilmington, DE (US)

(72) Inventors: Timothy Casper, Yorklyn, DE (US); Charles Philip Defelice, Wilmington, DE (US); David L. Hallahan, Wilmington, DE (US); Bruno Ieiri Ito, Sao Paulo (BR); Sharon Jo Keeler, Bear, DE (US); Katrina Kratz, Chadds Ford, PA (US); Markus Michael Liedgens, Sau Paulo (BR); Brian D. Mather, Newark, DE (US); Scott Meadows, Pennsville, NJ (US); Marcos Luciano Nunhez, Sao Paulo (BR); Joseph Anthony Perrotto, Landenberg, PA (US); Eduardo Da Cruz Maduro Picelli, Sao Paulo (BR); Laudenir Maria Prioli, Sao Paulo (BR); James John Ragghianti, Bear, DE (US); Christopher C. Whitcombe, Newark, DE (US)

(21) Appl. No.: 15/305,420

(22) PCT Filed: Apr. 23, 2015
(86) PCT No.: PCT/US15/27299

§ 371 Date: Oct. 20, 2016

Related U.S. Application Data
(60) Provisional application No. 61/984,093, filed on Apr. 25, 2014.

(51) Int. Cl. A01H 4/00; A01G 31/00; A01G 9/24; A01G 9/02; A01G 9/10; A01G 1/00; A01G 7/00

(52) U.S. Cl. CPC A01H 4/006; A01H 4/008; A01G 1/001; A01G 31/001; A01G 7/00; A01G 9/027; A01G 9/028; A01G 9/1026; A01G 9/24; A01G 2031/005

(57) ABSTRACT

The present disclosure provides a method for propagating a crop using meristem excision, micropropagation, plantlet formation, plantlet singulation, plantlet transfer to artificial seeds, and planting artificial seeds. Methods are provided for producing and handling the plantlets and materials used to form artificial seeds. The artificial seeds may be used to store plantlets or may be planted to form a crop.

### CROSS REFERENCE TO RELATED APPLICATIONS

This application claims the benefit of U.S. Provisional Application No. 61/984,093, filed Apr. 25, 2014, which is incorporated by reference in its entirety.

### FIELD

This disclosure relates to a process for the production and cultivation of plant artificial seeds.

### BACKGROUND

Sugarcane is an important crop worldwide. Sugarcane is a tropical, perennial crop with production of 26 MM Ha/year, yielding 1600 million tons of cane, equal to approximately 22% of total global crop production. The demand for sugarcane is increasing; yet current production methods limit the development and deployment of sugarcane varieties with improved characteristics like yield, disease resistance, herbicide resistance, or tolerance to abiotic stress. Current methods for propagating sugarcane entail cutting mature sugarcane stalks into smaller “seed cane” sections or “billets”. These billets are then planted in furrows in the field. One acre of mature cane is sufficient to plant approximately 5 acres. The harvesting and production/planting of seed cane is largely done manually, since the sectioning of mature cane is not easily mechanized. Billets are bulky, perishable and pack poorly. Logistics and timing for harvesting and planting billets is complex. Fungal and bacterial diseases are often transmitted or “carried over” in the seed cane. The cost of producing seed cane is approximately 24% of the cost of establishing a new crop. Approximately 19% of available arable land must be dedicated to nurseries producing seed cane. Varietal sugarcane choice is limited to planting material that has survived to maturity in the previous growing season. A typical production sugarcane field is grown and harvested over a 5 year period, therefore a 3-4 year lead time is required to introduce new varieties.

Another process for culturing sugarcane meristems into bud masses from field-grown stalks of sugarcane has been disclosed (BSES, WO2011/085446). This method allows for high multiplication factors, which can be used to accelerate variety release. However, the propagules from this process require hardening in a nursery before being transferred to the field, which limits their practicality for large scale sugarcane production.

There is interest and a need in the field of sugarcane production in moving to a more mechanized method for growing and planting this crop. Desirable features of a new paradigm for sugarcane would include a) less use of arable land for seed cane development, b) more rapid development and deployment of new sugarcane varieties, c) elimination of “carryover” disease when planting, d) fully mechanized planting, and e) less risk from environmental conditions during the development of seed cane.

### SUMMARY

The present disclosure provides methods and compositions to improve growth and viability of regenerable plant tissues and allows for a scaleable planting process of difficult to propagate plants, such as sugarcane.

A first aspect features a method of obtaining regenerable plant tissue, comprising a) excising meristems from plants, b) growing said meristems on solid support media in the presence of plant hormones, and c) growing the tissue from step (b) in liquid media in the presence of plant growth regulators (such as paclobutrazol) and/or plant hormones; wherein the tissue proliferates to a larger mass to form regenerable plant tissue.

In one embodiment, said step of growing the tissue from said step, growing said meristems on solid support media in the presence of plant hormones, in liquid media in the presence of plant growth regulators (such as paclobutrazol) and/or plant hormones is repeated at least once.

In another embodiment, said larger mass of regenerable plant tissue is subdivided, by steps comprising a) cutting the larger mass into smaller segments from about 1 mm³ up to about 1000 mm³, b) placing said fragments into temporary immersion with liquid medium for at least about 3-10 days, and c) replacing the medium after every about 3-10 days and continuing the culture expansion for up to 10 cycles. In another embodiment, tissue can be subdivided into additional temporary immersion units, as needed, throughout the culture process to preserve the tissue to media ratio. In another embodiment, regenerable tissue can be fragmented into smaller pieces at any time during the proliferation stage and moved to medium without hormones in which the tissue will form extended shoots and roots which can be separated into unique plantlets.

In another embodiment, said larger mass of regenerable plant tissue is subdivided using at least one of the following: chopping, automated slicing and/or grinding, or sieving, resulting in smaller regenerable plant tissue mass. This growth may be contained within a bioreactor and may further comprise fungicides, hormones, endophytes, and other plant growth stimulating compounds.

In another embodiment, a method of growing regenerable plant tissue is provided comprising a) placing the regenerable plant tissue into at least one of the following: i) a cavity of an array structure, ii) a tube like structure, iii) a support structure, or iv) a bioreactor; and b) growing the regenerable plant tissue wherein the regenerable plant tissue increases in mass. This method may be performed in liquid media with plant hormones to increase the mass of regenerable plant tissue.

In another embodiment, a method of growing regenerable plant tissue to form plantlets is provided, comprising a) placing the regenerable plant tissue into one of the following: i) a cavity of an array structure, ii) a tube like structure, iii) a support structure, or iv) a bioreactor, and; b) growing the regenerable plant tissue in media without plant hormones to form plantlets. The plantlets may be grown in, or transferred to, a tray structure or a plant artificial seed structure.

In one embodiment, there is at least a 100-×-fold amplification of plantlets for each meristem excised.

In another embodiment, there is at least a 1000-× fold amplification of plantlets for each meristem excised utilizing one or more methods of growing regenerable plant tissue.

In another embodiment, the amplified plantlets exhibit low levels of phenotypic abnormalities that can be attributed to somoclonal variation. Somoclonal variation may be less than between 0-10%, including less than 1%, 2%, 3%, 4%, 5%, 6%, 7%, 8%, 9%, or 10%.

In another embodiment, said liquid media is contained in a bioreactor.

In another embodiment, the media further comprises compounds selected from the group consisting of: antibiotics, fungicides, plant hormones, growth nutrients, salts, plant hormone inhibitors, growth regulators, agars, and insoluble materials.

In another embodiment, the media further comprises endophytes.

Another aspect features a method of separating individual plantlets from a larger mass of plantlets, comprising a) isolating a mass of plantlets, and subsequently b) exposing the mass to a blade-like structure, wherein individual plantlets are separated. Plantlets may also be manually separated into individual plantlets. Alternatively, the mass of plantlets may be exposed to a blade-like structure, or a belt and brush singulator, wherein individual plantlets are separated.

Another aspect features an array comprising a multi-cavity support for tissue growth to be used in continuous or temporary immersion liquid culture.

In one embodiment, the array cavities may be circular, square, rectangular or multi-sided shapes.

In another embodiment, the cavities are formed by machining, molding or any other suitable means such as welded honeycomb structures; and have a cavity diameter of about 1/16 to about ½ inch.

In another embodiment, the regenerable plant tissue is placed in said cavities by at least one of the following: manual placement, vibration distribution, vacuum using a transfer plate with suction patterns matching the cavity array pattern, distribution while floating in growth media, suspension in liquid followed by injection, or by grating, smashing or rollering tissue into array cavities. Fragments can also be distributed by rollering or smashing plant material into array cavities.

In another embodiment, said cavity has an upper and lower section separated by a tissue fragment support, the fragment support positioning the fragment vertically within the cavity creating shoot and root growth zones, wherein said shoot and root growth zones are of adequate thickness to allow plantlets to grow without becoming entwined with plantlets in adjacent cavities.

In another embodiment, the tissue fragment support prevents plantlets from falling out of the bottom of the cavity while allowing growth media to circulate vertically through the cavity.

In another embodiment, the plantlets are transferred directly from an array comprising a multi-cavity support for tissue growth to be used in continuous or temporary immersion liquid culture, to seed enclosures of the tray that is located on the same pitch or a multiple pitch of the cavity spacing.

In another embodiment, the transfer is accomplished by pushing the plantlet from the array into an artificial seed structure.

Another aspect features a method of forming plants from plantlets or regenerable plant tissue before transferring to a field, comprising exposing the plantlets to conditions that improve viability of the plants in said field, wherein the conditions include one or more of the following: a) increasing light intensity, b) eliminating added plant hormones, c) reducing humidity, and d) contacting plant roots with media supports such as Rockwool, Metro Mix 360 ®, soil, or other material.

One embodiment features a method of inserting the fully formed plants into artificial seed structures, comprising transferring plants from an array structure to a tray structure using a method selected from the group comprising, or alternatively consisting of, piston force transfer, vacuum transfer, sieving, manual transfer, and mechanical transfer.

Another aspect features a method of storing at least one plantlet placed within a structure, comprising one or more of: a) maintaining a moisture content within said seed structure of between 40%-80%, b) supplying a light source, and c) maintaining a temperature of at least 10° C. to 30° C., wherein the fully formed plants are stored to maintain viability for at least 1 week. The structures may also comprise media that contains hormones and/or hormone inhibitors. The structure may be, but is not limited to, a cavity of an array structure, a tube-like structure, or a support structure.

In an embodiment, the artificial seed structure comprises one or more compounds selected from the group comprising, or alternatively consisting of: soil, other rooting media, fungicides, nematicides, antibiotics, insecticides, antimicrobials, biocides, herbicides, plant growth regulator, plant hormone or growth stimulator, endophytes, molluscicides, miticides, acaricides, bird repellants, insect repellants, rodent repellants, growth nutrients, salts, plant hormone inhibitors, agar, and insoluble materials.

Another aspect features a method of transporting an artificial seed structure, comprising introducing an artificial seed structure into a space having one or more conditions selected from the group comprising, or alternatively consisting of: stacking trays, temperature controlled environments, humidity controlled environments, machinery to transfer artificial seed structures to a planter, machinery to transfer the artificial seed structures to transport vehicles, and transporting said seed structures.

Another aspect features a method of planting an artificial seed structure, comprising utilizing in an environment having one or more conditions selected from the group comprising, or alternatively consisting of: controlling the depth of the artificial seed structure in soil, addition of supplemental nutrients, separation of individual artificial seeds from tray structures, cutting of the artificial seed structure, orienting the artificial seed structure during planting, addition of agents to promote degradation of the artificial seed structure, machinery to transfer said artificial seed structure to the planter, and planting of said artificial seed structure.

In another embodiment, the seed structures are transported to a field and planted using mechanical means that ensure viability and vigor of the plants.

In another embodiment, the excised meristems, the regenerable plant tissue, and the plantlets are selected from the group consisting of: sugarcane, a graminaceous plant, Saccharum spp, Saccharum spp hybrids, Miscanthus, switchgrass, energycane, sterile grasses, orchids, cocoa, bamboo, cassava, corn, rice, banana, potato, sweet potato, yam, pineapple, trees, willow, pine trees, poplar, mulberry, Ficus spp, oil palm, date palm, poaceae, verbena, vanilla, tea, hops, Erianthus spp, intergeneric hybrids of Saccharum, Erianthus and Sorghum spp, African violet, apple, date, fig, guava, mango, maple, plum, pomegranate, papaya, avocado, blackberries, garden strawberry, grapes, canna, citrus, lemon, orange, grapefruit, tangerine, or dayap.

In another embodiment, the excised meristems, the regenerable plant tissue, and the plantlets are genetically modified.

Another aspect features a method of planting the plant contained within an artificial seed structure.

Another aspect features a method of planting a crop from excised meristems comprising: a) obtaining the excised meristems to form regenerable plant tissue, b) using the regenerable plant tissue of (a) to form plantlets, c) making the plants from the plantlets of (b), d) placing the plantlets of (b) or the plants of (c) into an artificial seed structure, and e) planting the artificial seed structure of (d) to obtain a crop.

Another aspect features a method of planting sugarcane, said method comprising: (i) obtaining a regenerable plant tissue comprising, a) growing excised sugarcane meristem tissue on solid support media in the presence of plant hormones, and b) transferring the tissue from step a) to liquid growth media in the presence of plant growth regulators; and growing to form the regenerable plant tissue; (ii) subdividing said regenerable plant tissue comprising, a) cutting said tissue into smaller segments having a size from about 1 mm³ up to about 1000 mm³, b) placing said cut tissue into temporary immersion with liquid medium for at least about 3-10 days, and c) replacing the medium after about every 3-10 days; (iii) further growing said subdivided regenerable plant tissue, comprising, a) placing the plant tissue into at least one of the following: a cavity of an array structure, a tube-like structure, or a support structure; and b) growing said tissue; (iv) forming plantlets from said regenerable plant tissue by growing in media lacking hormones; (v) transferring the plantlets from the structures of (iii) to an artificial seed; (vi) forming plants from said plantlets, comprising exposing the plantlets to one or more of the following conditions: a) increased light intensity, b) reduced humidity, and c) contact of plant roots with media supports such as Rockwool, MetroMix®360, soil or other material; (vii) transporting said artificial seed structure with said plants, comprising, a) introducing said artificial seed structure into a space, and b) transporting said seed structures to a field; and (viii) planting the artificial seed structure.

Another aspect features a method of planting sugarcane, said method comprising: (i) obtaining a regenerable plant tissue comprising, a) growing excised sugarcane meristem tissue on solid support media in the presence of plant hormones, and b) transferring the tissue from step a) to liquid growth media in the presence of plant growth regulators; and growing to form the regenerable plant tissue; (ii) subdividing said regenerable plant tissue comprising, a) cutting said tissue into smaller segments having a size from about 1 mm³ up to about 1000 mm³, b) placing said cut tissue into temporary immersion with liquid medium for at least about 3-10 days, and c) replacing the medium after about every 3-10 days; (iii) further growing said subdivided regenerable plant tissue in an artificial seed structure; (iv) forming plantlets from said regenerable plant tissue by growing in media lacking hormones; (v) forming plants from said plantlets, comprising exposing the plantlets to one or more of the following conditions: a) increased light intensity, b) reduced humidity, and c) contact of plant roots with media supports such as Rockwool, MetroMix®360, soil or other material; (vi) transporting said artificial seed structure with said plants, comprising, a) introducing said artificial seed structure into a space, and b) transporting said seed structures to a field; and (vii) planting the artificial seed structure.

In one embodiment, said step of growing excised sugarcane meristem tissue on solid support media in the presence of plant hormones is repeated at least once.

In another embodiment, said regenerable plant tissue in step (ii) is subdivided using at least one of the following: chopping, automated slicing, automated grinding, or sieving. In another embodiment, subdividing said regenerable plant tissue comprises cutting said tissue into smaller segments having a size from about 1 mm³ up to about 1000 mm³. Segment size may be between 1 mm³ up to about 1000 mm³, including any value between 1 mm³ and 1000 mm³.

In another embodiment, there is at least a 1000-×-fold amplification of plantlets for each meristem excised.

In another embodiment, the media further comprises compounds selected from the group consisting of: antibiotics, fungicides, plant hormones, growth nutrients, salts, plant hormone inhibitors, growth regulators, agars, and insoluble materials. In another embodiment, the media further comprises endophytes.

In another embodiment, the array comprises a multi-cavity support for tissue growth used in continuous or temporary immersion tissue culture.

In another embodiment, the array cavities are circular, square, rectangular, or multishaped.

In another embodiment, the array cavities are formed by machining, molding or any other suitable means such as welded honeycomb structures; and have a cavity diameter of about 1/16 to ½ inch.

In another embodiment, the regenerable plant tissue is placed in said array cavities by at least one of the following: manual placement, vibration distribution, vacuum using a transfer plate with suction patterns matching the cavity array pattern, distribution while floating in growth media, suspension in liquid followed by injection, or by grating, smashing or rollering tissue into array cavities.

In another embodiment, said array cavity or artificial seed structure has an upper and lower section separated by a tissue fragment support, the fragment support positioning the fragment vertically within the cavity creating shoot and root growth zones, wherein said shoot and root growth zones are of adequate thickness to allow plantlets to grow without becoming entwined with plantlets in adjacent cavities.

In another embodiment, inserting the plantlet into the artificial seed structure comprises transferring said plantlet from an array structure to a tray structure using a method selected from the group consisting of piston force transfer, vacuum transfer, sieving, manual transfer, and mechanical transfer.

In an embodiment, said artificial seed structure is stored in conditions comprising

maintaining a moisture content within said seed structure of between 40%-80%, supplying a light source, and maintaining a temperature of at least 10° C. and 30° C. wherein the fully formed plants remain viable for at least one week.

In another embodiment, the artificial seed structure comprises compounds selected from the group consisting of: soil, other rooting media, fungicides, nematicides, antibiotics, insecticides, antimicrobials, biocides, herbicides, plant growth regulator or stimulator, endophytes, mollucicides, miticides, acaricides, bird repellants, insect repellants, rodent repellants, growth nutrients, salts, plant hormone inhibitors, agar, and insoluble materials.

In another embodiment, the excised meristems, the regenerable tissue, and the plants are genetically modified.

In another embodiment, said space comprises stacking trays.

In another embodiment, said space is a temperature controlled environment.

In another embodiment, said space comprises machinery to transfer said artificial seed structures to a planter.

In another embodiment, said space comprises machinery to transfer said artificial seed to a transport vehicle.

In another embodiment, said planting comprises controlled planting depth.

In another embodiment, said planting comprises addition of supplemental nutrients.

In another embodiment, said planting comprises separation of individual artificial seeds from tray structures.

In another embodiment, said planting comprises cutting of the artificial seed structure.

In another embodiment, said planting comprises orienting the artificial seed structure during planting.

In another embodiment, said planting comprises addition of degradation promoting agents.

In another embodiment, said planting comprises machinery to transfer said artificial seed structures to a planter.

In another embodiment, said planting is in a field.

### BRIEF DESCRIPTION OF THE FIGURES

The disclosure can be more fully understood from the following detailed description and the accompanying drawings which form a part of this application.

FIG. 1 shows a representative micropropagation process for sugarcane described herein. Variations of this process exist and are not limited to the process described in FIG. 1.

FIG. 2 shows a representative micropropagation, seed assembly, and planting process for sugarcane described herein. Variations of this process exist and are not limited to the process described in FIG. 2.

FIG. 3 shows a second, representative micropropagation, seed assembly, and planting process for sugarcane described starting with fragments at Stage 2.2 Variations of this process exist and are not limited to the process described in FIG. 3.

FIG. 4 shows height (cms) to the first dewlap of micropropagation-derived sugarcane plants from the day of planting until 42 days after planting. Cont=control; Cont+N=Control+added nitrate; A. d.=Acetobacter diazotrophicus ATCC#49037; H. s.=Herbaspirillum seropedicae ATCC#35892; A.d.+H.s.=mixture of two strains

FIG. 5 shows the growth of micropropagated plants from 6 weeks to 10 weeks after inoculation with endophytes Herbaspirillum seropedicae ATCC#35892 and Pantoea agglomerans #100.

FIG. 6 exemplifies one array structure: (1) is the media screen; (2) is the bioreactor array; (3) is the handle; (4) is the bottom support; (5) and (6) are nuts and bolts to fasten the components together.

FIG. 7 shows resultant sugarcane plantlets regenerated after four weeks of this experiment. Plantlets were generated from each of the hole sizes with percentage of viable plants proportionate to hole size.

FIG. 8 shows the arrangement of a 1000 cavity array structure: (1) is the post; (2) is the bioreactor array; (3) is the media screen; (4) is the bottom support; (7) is the alignment post and (9) is a stainless steel nut.

FIG. 9 shows the resultant plantlets regenerated after four weeks.

FIG. 10 exemplifies another array structure. (1) array; (2) handle; (3) root array block; (4) media screen; (5) thumb nuts and (6) stainless steel bolts.

FIG. 11 shows the plantlets regenerating after one week of a four week experiment.

FIG. 12 shows the length (height) of plants to the dewlap structure when grown in potting mix in a greenhouse setting. These plants were derived from bioreactors (RITA) versus array structures in bioreactors (Forms). DAP=Days after planting

FIG. 13 shows the number (#) of plant stalks when plants derived from in bioreactors (RITA) versus array structures in bioreactors (Forms) were planted into potting mixture in a greenhouse setting. DAP=Days after planting

FIG. 14 shows the percent survival after transplantation to the field of plants grown in bioreactors with and without arrays. Three different experimental results are shown (Experiments A to C).

FIG. 15 shows percent of regenerating fragments that emerged from wells on Rockwool. RW=Rockwool

FIG. 16 shows regeneration of fragments into plants on various substrates in static tube bioreactors. Regeneration and plant growth was scored following 1 week of growth in the static tube bioreactor at a light level of 46 μmol/m²/sec and an additional 1 week in non-sterile conditions at 250 μmol/m²/sec. Each substrate was tested with 9-11 fragments.

FIG. 17 shows a mold design for a top portion of a tray assembly.

FIG. 18 shows a mold design for a bottom portion of a tray assembly.

FIG. 19 shows a plug assist for the top tray mold.

FIG. 20 shows effects of different components on the degradation of cellulosic composites considering a confidence level of 95%. The standardized effect was calculated from the difference between the average degradation response for all samples and the response for each sample with that factor. The value 2.120 separates the factors that are significant from those that presented a very low effect and takes into account the 95% confidence interval for all samples.

FIG. 21A-D shows a short straw experiment from plant regeneration to encapsulation. FIG. 21A shows 7 days of regeneration in TIS, at phase 3.1. FIG. 21B shows optional 7 days of regeneration in sterile condition, at phase 3.2. FIG. 21C shows plants fully developed and hardened in the greenhouse, phase 4. FIG. 21D shows a tube-like plastic structure for encapsulation, phase 5. FIGS. 21A-B shows 7 to 14 days in vitro. FIG. 21C shows 14 to 21 days greenhouse. FIG. 21D shows 14 days greenhouse.

FIG. 22 shows a schematic illustration of the short, tube-like structure at the first two weeks of plant regeneration from micropropagated fragments.

FIG. 23 shows an interval plot of the efficiencies throughout the tube-like process. Survival rates of plants regenerated in the successive 4 phases of the small, tube like structure process (see FIG. 21A-D), before planting. In these experiments, tube-like structures were 8 mm diameter and 60 mm length. The in vitro regeneration comprising Phase 3.1 (7 days) and the optional Phase 3.2 (additional 7 days). Phase 4 corresponds to the hardening of the tube-like structure in the greenhouse. Phase 5 corresponds to the assembling of the original tube-like structure into a final structure for encapsulation. 95% CI for the mean

FIG. 24A-B shows a summary for advantages of the short, tube like structure. FIG. 24A shows Type 3 plants in the greenhouse at 4 weeks of regeneration. FIG. 24B shows 4 week old Type 3 plants in the greenhouse and ready for encapsulation into the final structure for direct transfer to the field.

FIG. 25 shows survival rate of artificial seeds of sugarcane during the first 90 days after transplanting to the field. Three variations of the regeneration process with tube-like structures (Types 1 to 3) and a control with standard regeneration (temporary immersion) are displayed (see Example 17).

### DETAILED DESCRIPTION

The present disclosure relates to a process for forming proliferated, regenerable plant tissue from excised meristematic tissue, forming plantlets from the regenerable plant tissue, and packaging and planting the plantlets to form a crop. In one embodiment, the present disclosure relates to a process for sugarcane micropropagation from excised meristematic tissue, artificial seed structure assembly, sugarcane storage and transport and planting.

The present disclosures will now be described more fully hereinafter with reference to the accompanying drawings, in which some, but not all embodiments of the disclosures are shown. Indeed, these disclosures may be embodied in many different forms and should not be construed as limited to the embodiments set forth herein. Like numbers refer to like elements throughout.

Descriptions of various embodiments of the process are set forth.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes, and symbols are denoted in their International System of Units (SI) accepted form. Unless otherwise indicated, nucleic acids are written left to right in 5′ to 3′ orientation; and amino acid sequences are written left to right in amino to carboxy orientation. Numeric ranges recited within the specification are inclusive of the numbers defining the range and include each integer within the defined range. Nucleotides may be referred to herein by their one-letter symbols recommended by the IUPAC-IUBMB Nomenclature Commission. The terms defined below are more fully defined by reference to the specification as a whole. Section headings provided throughout the specification are provided for convenience and are not limitations to the various objects and embodiments of the present disclosure.

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein, the term “comprising” means “including but not limited to.”

As used herein, cane top refers to the top part of a sugarcane stalk removed from the intact plant and then used to harvest the explant.

As used herein, explant refers to, generically, the tissue removed from a plant and introduced into culture.

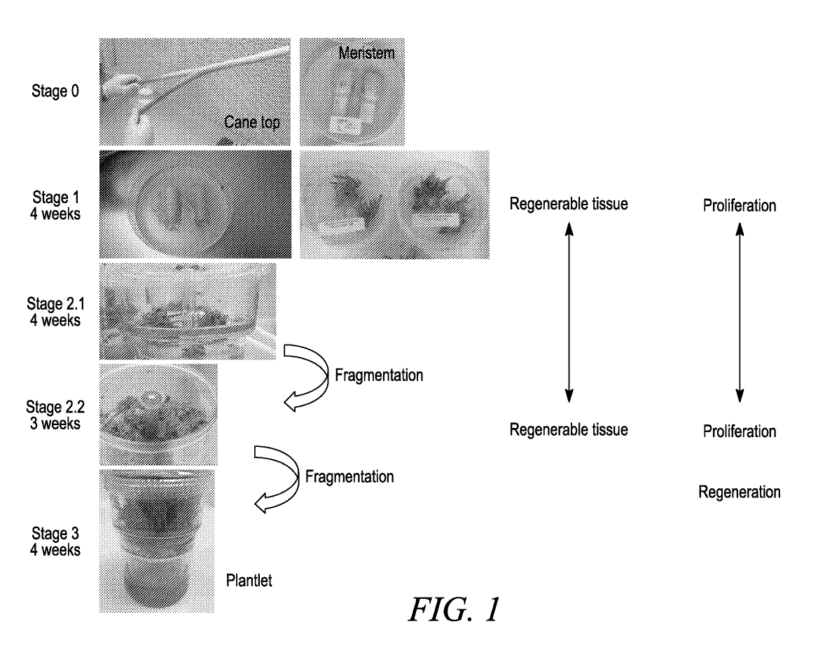

As used herein, meristem (such as, but not limited to, bud or apical meristem) refers to the apical (terminal) shoot bud or primordia or the axial primordia that is removed from a cane top in the described process and introduced into culture. See, for example, FIGS. 1-3.

As used herein, regenerable tissue (such as, but not limited to, proliferated bud tissue) refers to plant meristematic tissue that has been introduced into tissue culture and continuing until the time it is placed onto regeneration media (for example, without cytokinin). Regenerable plant tissue is plant tissue with meristematic properties capable of initiating shoot formation, proliferation of multiple meristem initials, resulting in an increase in plant tissue mass. Regenerable plant tissue can lead to plantlet formation when cultured in the absence of plant hormones, such as cytokinin. This growth may be contained within a bioreactor and may further comprise fungicides, hormones, endophytes, and other plant growth stimulating compounds.

As used herein, fragment refers to a piece of regenerable tissue derived from a larger mass and created by fragmentation. Larger masses of regenerable plant tissue can be subdivided using at least one of the following methods: chopping, automated slicing and/or grinding, or sieving, resulting in smaller regenerable plant tissue mass.

As used herein, plantlets refer to structures developing shoots and roots, produced in culture utilizing media lacking cytokinin.

As used herein, plant refers to a structure that is established and growing in soil in an open atmosphere. A plantlet converts into a plant when it is placed into soil in a field, greenhouse, growth chamber or screenhouse and grown without a dome or other covering. A plantlet inside a seed would transition to a plant as it emerges from the seed structure.

As used herein, sod refers to a mass of closely spaced and intertwined plantlets.

As used herein, dewlap refers to the topmost one of a pair of hinges at the joint of a sugarcane leaf blade and is used as the standard point for growth measurements.

As used herein, proliferation refers to the process of growth and increase in size and number of meristems of the regenerable tissue during stages 1, 2.1, 2.2, 2.3, etc of the standard process (see, for example, FIGS. 1-3). The various stages referred to throughout this disclosure are made with respect to the stages set forth in FIG. 1, for example.

As used herein, trimming refers to the process of removing shoots, leaves, and/or roots, from a regenerable tissue or a plantlet or a plant. This may be done in stages 1, 2, 3, during seed assembly, and after storage.

As used herein, regeneration refers to the process of converting regenerable tissue into plantlets with roots and extended shoots, for example, during stage 3 tissue culture (for example, incubation without cytokinin).

As used herein, fragmentation refers to the process of converting a large mass of regenerable tissue into multiple smaller units or fragments. This can be done with any stage 2 tissue (eg 2.1, 2.2, 2.3, etc.). Fragmentation can be done by various processes including use of machinery, hand dicing, or other means. Self-disaggregation may occur without human intervention as tissue matures. Self-disaggregation refers to the process whereby masses of regenerable tissue separate from each other as they regenerate into multiple plantlets. This occurs without extra human intervention during stage 3.

As used herein, singulation refers to the process of producing, from a mass of regenerable tissue or plantlets, a single unit that either is a plantlet or is regenerable tissue which can be regenerated into a plantlet. Once singulated, the unit can then be manipulated individually, for example inserted into an array (for regenerable tissue) or into a tray (for plantlets). Singulation of plantlets can be done prior to inserting them into an artificial seed structure (such as in International Publication No. WO 2013/096531, incorporated by reference herein) or an array.

As used herein, an array is any support with cavities which allow insertion of fragments or plantlets to isolate them from each other and determine the plant tissue contributing to a unit of final product.

The terms artificial seed structure, artificial seed, and synthetic seed structure may be used interchangeably, as used herein. An artificial seed of the present invention comprises a container and a regenerable plant tissue. Artificial seeds are described in International Publication Nos. WO 2013/096531 and WO 2013/096536, both which are incorporated by reference herein.

In an embodiment, the artificial seed structure comprises one or more compounds selected from the group comprising, or alternatively consisting of: soil, other rooting media, fungicides, nematicides, antibiotics, insecticides, antimicrobials, biocides, herbicides, plant growth regulator, plant hormone or growth stimulator, endophytes, molluscicides, miticides, acaricides, bird repellants, insect repellants, rodent repellants, growth nutrients, salts, plant hormone inhibitors, agar, and insoluble materials.

As used herein, storage refers to maintaining any material (for example, regenerable tissue, fragments, plantlets, seeds, plants) in a condition where the primary purpose is to hold the material in a static condition assisting progress to a subsequent stage.

As used herein, hardening (included but not limited to acclimation, acclimatization, adaptation) refers to treatment of regenerable tissue, plantlets or plants in a manner designed to make them better able to survive and grow when placed into field conditions. Hardening can occur during proliferation, but more likely during regeneration, or storage.

As used herein, assembly refers to the process of inserting a plantlet and media and optionally other components into an enclosure to create a seed or an array of seeds.

As used herein, sowing refers to the process of transferring seeds into soil in a field or in a pot in a greenhouse, growth chamber, screenhouse, in such a way that they will survive and grow into mature plants. This can be done by hand or by a mechanical planter.

As used herein, separation refers to the process of separating, such as a tray of seeds into single seeds ready for planting.

Another aspect of the disclosure features a method of separating individual plantlets from a larger mass of plantlets, comprising a) isolating a mass of plantlets, and subsequently b) exposing the mass to a blade-like structure, wherein individual plantlets are separated. Plantlets may be manually separated into individual plantlets. Alternatively, the mass of plantlets may be exposed to a blade-like structure, or a belt and brush singulator, wherein individual plantlets are separated.

### Micropropagation Process

An aspect of the disclosure features a method of planting sugarcane, said method comprising (i) obtaining a regenerable plant tissue comprising, a) growing excised sugarcane meristem tissue on solid support media in the presence of plant hormones, and b) transferring the tissue from step a) to liquid growth media in the presence of plant growth regulators; and growing to form the regenerable plant tissue; (ii) subdividing said regenerable plant tissue comprising, a) cutting said tissue into smaller segments having a size from about 1 mm³ up to about 1000 mm³, b) placing said cut tissue into temporary immersion with liquid medium for at least about 3-10 days, and c) replacing the medium after about every 3-10 days; (iii) further growing said subdivided regenerable plant tissue, comprising, a) placing the plant tissue into at least one of the following: a cavity of an array structure, a tube-like structure, or a support structure; and b) growing said tissue; (iv) forming plantlets from said regenerable plant tissue by growing in media lacking hormones; (v) transferring the plantlets from the structures of (iii) to an artificial seed; (vi) forming plants from said plantlets, comprising exposing the plantlets to one or more of the following conditions: increased light intensity, reduced humidity, and contact of plant roots with media supports such as Rockwool, MetroMix®360, soil or other material; (vii) transporting said artificial seed structure with said plants, comprising, a) introducing said artificial seed structure into a space, and b) transporting said seed structures to a field; and (viii) planting the artificial seed structure.

Another aspect of the disclosure features a method of planting sugarcane, said method comprising: (i) obtaining a regenerable plant tissue comprising, a) growing excised sugarcane meristem tissue on solid support media in the presence of plant hormones, and b) transferring the tissue from step a) to liquid growth media in the presence of plant growth regulators; and growing to form the regenerable plant tissue; (ii) subdividing said regenerable plant tissue comprising, a) cutting said tissue into smaller segments having a size from about 1 mm³ up to about 1000 mm³, b) placing said cut tissue into temporary immersion with liquid medium for at least about 3-10 days, and c) replacing the medium after about every 3-10 days; (iii) further growing said subdivided regenerable plant tissue in an artificial seed structure; (iv) forming plantlets from said regenerable plant tissue by growing in media lacking hormones; (v) forming plants from said plantlets, comprising exposing the plantlets to one or more of the following conditions: increased light intensity, reduced humidity, and contact of plant roots with media supports such as Rockwool, MetroMix®360, soil or other material; (vi) transporting said artificial seed structure with said plants, comprising, a) introducing said artificial seed structure into a space, and b) transporting said seed structures to a field; and (viii) planting the artificial seed structure.

As shown in FIG. 1, sugarcane micropropagation may begin with an excised apical meristem (Stage 0), solid phase culture of the meristem tissue for 1-4 weeks (Stage 1), and proceed through an initial round of micropropagation in a temporary immersion bioreactor (Stage 2.1), repeating the regenerable plant tissue proliferation in a temporary immersion bioreactor (Stage 2.2), and conclude with the formation of a sugarcane plantlet (Stage 3) in temporary immersion or static bioreactor or directly in rooting media. Variations of this process exist and are not limited to the process described in FIG. 1.

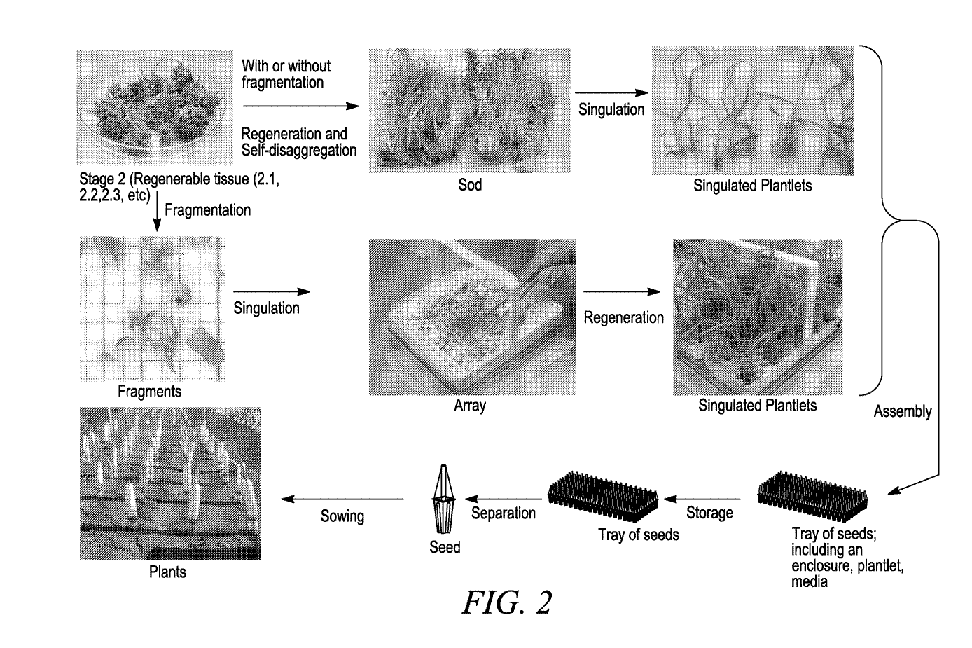

As shown in FIG. 2, regenerable plant tissue at different stages may be used for the process described herein. The regenerable plant tissue may be grown in different culture conditions to form fragments, sod, or arrays, which can be singulated manually, or by growth in array structures, and packaged in a variety of structures and materials for the purpose of storage and planting. Variations of this process exist and are not limited to the process described in FIG. 2.

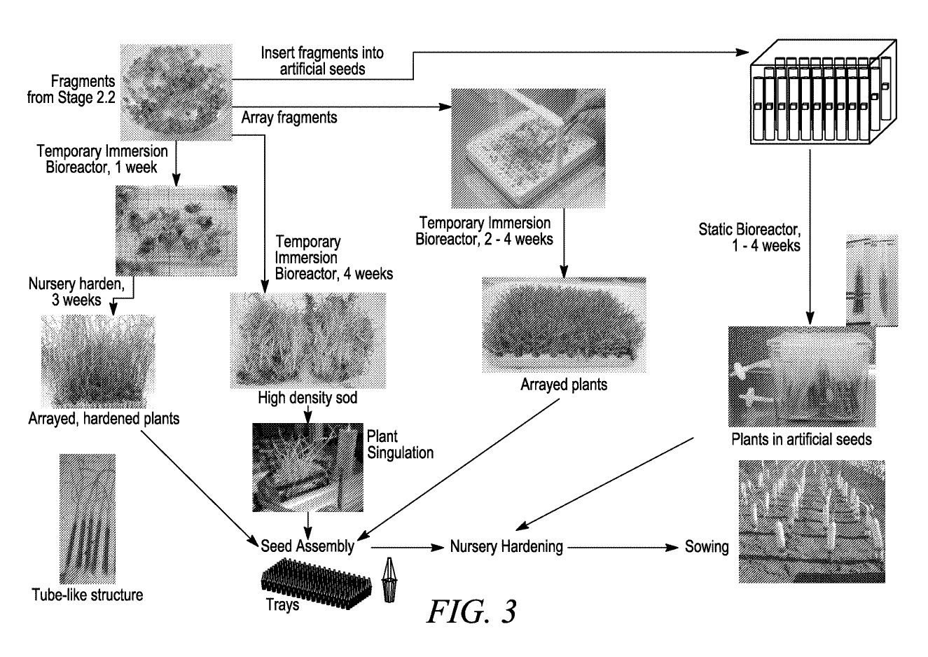

As shown in FIG. 3, fragments from Stage 2.2 may be used for the process described herein. The fragments may be grown in different structures, such as arrays, trays, or tube-like structures. Variations of this process exist and are not limited to the process described in FIG. 3.

In one embodiment, there is at least a 100-×-fold amplification of plantlets for each meristem excised.

In another embodiment, there is at least a 1000-× fold amplification of plantlets for each meristem excised utilizing one or more methods of growing regenerable plant tissue. In another embodiment, the amplified plantlets exhibit low levels of phenotypic abnormalities that can be attributed to somoclonal variation. Somoclonal variation may be less than between 0-10%, including less than 1%, 2%, 3%, 4%, 5%, 6%, 7%, 8%, 9%, or 10%.

### Materials and Structures

In an embodiment, a method of growing regenerable plant tissue is provided comprising a) placing the regenerable plant tissue into at least one of the following: i) a cavity of an array structure, ii) a tube-like structure, iii) a support structure, or iv) a bioreactor; and b) growing the regenerable plant tissue wherein the regenerable plant tissue increases in mass. This method may be performed in liquid media with plant hormones to increase the mass of regenerable plant tissue.

In another embodiment, a method of growing regenerable plant tissue to form plantlets is provided, comprising a) placing the regenerable plant tissue into one of the following: i) a cavity of an array structure, ii) a tube-like structure, iii) a support structure, or iv) a bioreactor, and; b) growing the regenerable plant tissue in media without plant hormones to form plantlets. The plantlets may be grown in, or transferred to, a tray structure or a plant artificial seed structure.

Tube-like structures comprise hollow, cylindrical structures that may be used for containing a plant material. In an embodiment, tube-like structures, include but are not limited to, straws. Tube-like structures may have variable lengths and widths. For example, dimensions may include, but are not limited to, about 3 to 12 mm diameter and about 40 to 70 mm length.

Using short, tube-like structures may be advantageous for several reasons. In vitro culture time may be reduced to 7 days (vs 28 days) with lower costs and less chance for genetic mutations. Production of individualized plants allows automation. Significantly improved plant rooting and root quality may result. Plant hardening before encapsulation into final structures for planting in the field may occur. Reduced required greenhouse space for hardening results in addition to reduced plant injuries for encapsulation. Plants are more protected; therefore the risks of damage by manipulation are minimized.

As used herein, an array refers to a group of cavities or cells that may be used for plantlet regeneration during, for example, stage 3 and that can be handled as a group. The cavities can be physically connected together (eg. as in the arrays used in Plant Forms bioreactors) or can be held together in some way. Arraying fragments, as described herein may provide several advantages, such as but not limited to, improving the mechanical process of transferring plants into seed structures using mechanical means.

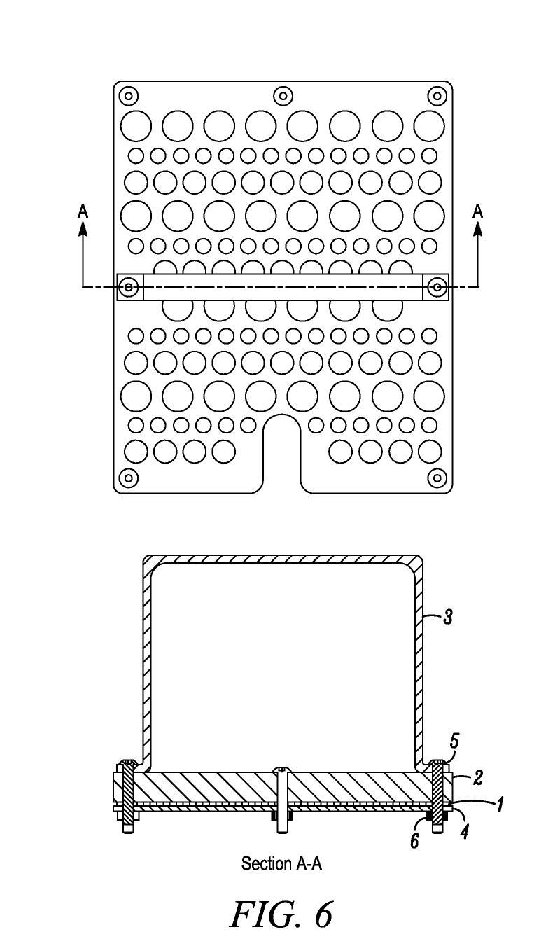

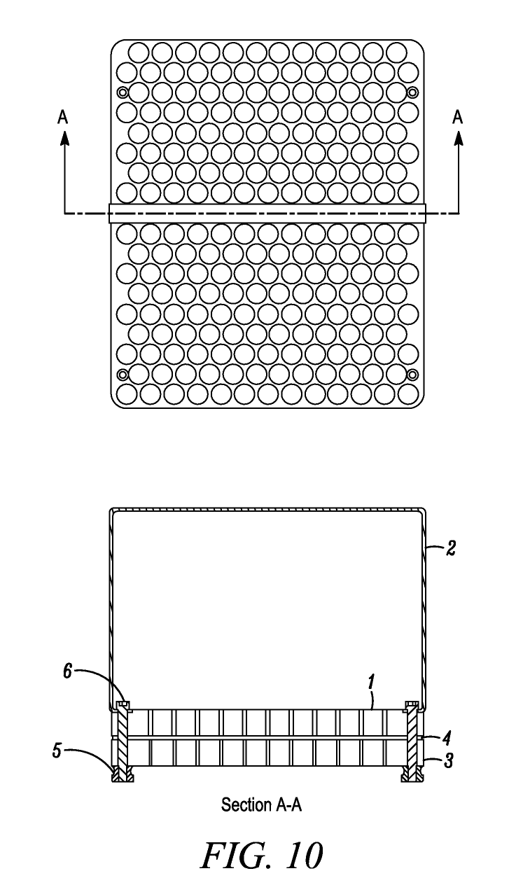

As described herein, FIG. 6, FIG. 8, and FIG. 10 exemplify three different array structures. For example, FIG. 8 shows the arrangement of a 1000 cavity array structure. The array structure is not limited to those shown in FIG. 6, FIG. 8, or FIG. 10.

One aspect of the disclosure features an array comprising a multi-cavity support for tissue growth to be used in continuous or temporary immersion liquid culture. In another embodiment, the array cavities may be circular, square, rectangular or multi-sided shapes. Further, the cavities are formed by machining, molding or any other suitable means such as welded honeycomb structures; and have a cavity diameter of about 1/16 to about ½ inch. The regenerable plant tissue may be placed in said cavities by at least one of the following: manual placement, vibration distribution, vacuum using a transfer plate with suction patterns matching the cavity array pattern, distribution while floating in growth media, suspension in liquid followed by injection, or by grating, smashing or rollering tissue into array cavities. Fragments can also be distributed by rollering or smashing plant material into array cavities.

In another embodiment of the disclosure, said cavity has an upper and lower section separated by a tissue fragment support, the fragment support positioning the fragment vertically within the cavity creating shoot and root growth zones, wherein said shoot and root growth zones are of adequate thickness to allow plantlets to grow without becoming entwined with plantlets in adjacent cavities. Further, the tissue fragment support prevents plantlets from falling out of the bottom of the cavity while allowing growth media to circulate vertically through the cavity.

In another embodiment, the plantlets are transferred directly from an array comprising a multi-cavity support for tissue growth to be used in continuous or temporary immersion liquid culture, to seed enclosures of the tray that is located on the same pitch or a multiple pitch of the cavity spacing. The transfer may be accomplished by pushing the plantlet from the array into an artificial seed structure.

Support layers contained within arrays include, but are not limited to, coarse screens, thin films, bagasse, soil, potting mixes, and sand. As an alternative to the coarse screen, a film can be sandwiched between the shoot and root zone plates. The film may be cut in registration with each cavity to form a series of flaps that support the fragment, provide an opening for the roots to grow and easily deflect to allow the plantlet to be transferred from the support without disassembly.

Another alternative to the coarse screen is to provide a series of flexures that protrude into the cavity opening to support the tissue and can be easily displaced for plantlet transfer. These flexures may be incorporated into a thin sheet that is sandwiched between the support plates similar to the slit film or molded integral to a single piece support. In the array, the base of the support is positioned vertically above the base of the tissue culture vessel to provide adequate growth space for roots. This space may be created by protrusions included in the bottom of the support structure or ledges in the tissue culture vessel that position the tissue support at the proper elevation when it is placed in the tissue culture vessel. A lifting means may be provided to easily remove the support from the vessel.

The support may be comprised of smaller modular sections that can be handled individually and then placed into the tissue culture vessel to facilitate aseptic processing. The support may be made of materials suitable for sterilization by means including autoclaving, irradiation or gas sterilization.

There are other means to achieve temporary immersion in an array without having a separate screen, film, filter, etc. Instead of having the cavities completely drilled through or by some other fabrication means, the array may feature a perforated lower surface with several tiny holes under each cavity. In this way, the array is a single part rather than an assembly of individual parts. In another embodiment, the array includes a completely solid floor supporting the plant tissue fragments with a series of channels or holes drilled through from the sides slightly above its base. In this embodiment, the channels, like the screen, are smaller than the size of the fragment (no larger than ⅛″ in diameter).

As used herein, tray refers to a collection of cavities or cells or enclosures or seeds that can be used to construct a group of artificial seed structures. A tray can also refer to a group of artificial seed structures after they are assembled. The tray allows the cavities to be handled as a group and, in one embodiment, may be associated together. For example, an empty or unassembled tray could be plug trays, or 200 well trays. An assembled tray could be two plug trays, sealed together with plantlets and media inside.

As used herein, an enclosure refers to a material that encloses a cavity that contains the plantlet and the media and optionally other components, which together comprise the seed. The enclosure can be made from more than one component (eg 2 plastic trays glued together) and may contain openings.

As used herein, seed refers to a single enclosure containing at a minimum a plantlet and media and that can be sown and grown into a plant. A seed may also contain growth promoting materials (pesticides, beneficial organisms, superabsorbent polymers, plant growth regulators, etc.).

As used herein, cytokinin refers to any of a number of different chemicals that have activity generally associated with cytokinins in plants. Cytokinins include not just 5-benzylamino purine (BAP), but also other chemicals as well. Cytokinins include, but are not limited to, 6-Benzylaminopurine, zeatin ((4-hydroxy-3-methyl-trans-2butenylaminopurine), 2-isopentenyladenine, (6-(4-hydroxy-3-methyl-trans-2-butenyl), zeatin-riboside)), kinetin (6-furfurylaminopurine, CPPU (N-(2-chloro-4-pyridyl)-N′-phenylurea), 1,3-diphenylurea, thiadiazuron (THZ), and DA-6 (diethyl aminoethyl hexanoate).

As used herein, soil refers to a generic term for a growing substrate for plantlets and plants consisting of sand, silt, clay, peat moss, other organic matter, vermiculite, perlite, fertilizer, etc. Soil can be native (for example, taken from a natural setting like a field) or artificial (for example, assembled from various natural or man-made ingredients) or a mixture of native and artificial. Soil includes metro-mix, jiffy, vermiculite, sand, Matapeake, field soils, etc.

As used herein, media (medium) refers to materials used to support growth of regenerable tissue and plantlets in culture or inside seeds. Media components can include sucrose, water, agar, salts, fertilizer, MS medium, plant growth regulators, hormones, antibiotics, soil, Rockwool, vermiculite, etc. Media can be sterile (for tissue culture applications) or non-sterile (used inside seeds).

As used herein, a bioreactor refers to a device for incubating plant material under sterile tissue culture conditions to allow it to grow. The term bioreactor is usually only used for devices where the medium is liquid. For example a petri plate containing agar media would not be termed a bioreactor. However, a container with rock wool saturated with liquid medium could be considered a bioreactor. Variations include temporary immersion bioreactor (TIB), static bioreactor, and the like.

As used herein, the term plant includes plant cells, plant organs, plant protoplasts, plant cell tissue cultures from which plants can be regenerated, plant calli, plant clumps and plant cells that are intact in plants or parts of plants such as embryos, pollen, ovules, seeds, leaves, flowers, branches, fruit, stalks, roots, root tips, anthers, and the like. Mature seed produced may be used for food, feed, fuel or other commercial or industrial purposes or for purposes of growing or reproducing the species. Progeny, variants and mutants of the regenerated plants are also included within the scope of the disclosure.

### Tray Shape and Fabrication

In one embodiment, the tray shape comprises a plurality of cavities, connected in a grid type arrangement. In this embodiment, an upper tray is used to make a top portion of the seed and a lower tray is used to make a bottom portion. The bottom portion contains media and the root and base stem portion of the plantlet. The top portion contains an airspace, in addition to the shoots of the plantlet. In this embodiment, the shape of the cavities differs between the trays used to make the top and bottom portions.

In another embodiment, the top portion possesses a tapered shape, leading to a single, central hole. This may comprise a frusto-conical shape, or a pyramidal shape. The bottom portion possesses a conical or tapered shape. The bottom of this shape may be flat or conical. The bottom tray will have either one or a plurality of holes which allow the roots of the plantlet to escape as they grow. The cross sectional shape of the tray-based seeds may be circular, square, rectangular, square with rounded corners, hexagonal or any desired cross-section. The gap between the cavities in the trays in this embodiment is between about 0.5-25 mm. The connections between the cavities of the trays may be perforated or otherwise weakened in order to facilitate separation of the seeds during planting.

In an assembled seed structure, the two trays may be held together by one or more of the following means: heat sealing, RF sealing, ultrasonic welding, an adhesive or wrapping the joint with a tape or film. Another means of joining the trays is through complementary “snap-together” surface features.

Trays may be fabricated through thermoforming of sheets. Methods of thermoforming include but are not limited to those described in “Technology of Thermoforming” Throne, J. L. 1996. Sheets may be fabricated by methods known in the art, such as extrusion and casting from the melt. The material used to make the sheets will be either resin pellets or powder, or mixtures thereof. Sometimes, the use of multiple types of pellets will be advantaged. The sheet may also consist of multiple layers of material with different compositions. The sheet may have thicknesses ranging from about 100 um to about 2500 um and may be on a roll or consist of individual square or rectangular formed sheets. The thermoforming process entails heating of the sheet to soften it, placing of the sheet on top of a negative (female) mold surface, deforming of the sheet to match the mold surface, trimming of the sheet, and removal from the mold. The sheet may be heated using methods known in the art, including radiant and convection heaters. The sheet may be gripped mechanically in order to move it through the ovens and onto the mold. In placing the sheet over the mold, a seal will be formed to allow vacuum to be applied. Applying vacuum from inside the mold as well as possibly air pressure and/or a mechanical “plug assist” from above will deform the sheet and shape it to match the mold. The plug assist may also possess holes through which air pressure may be applied. The formed part will then be cooled to harden it and trimmed to remove excess material, with the final part removed from the mold. The mold and plug assist may optionally be heated, or cooled. A release agent may be used to aid in removing the final part from the mold. The release agent may consist of one or more of several compounds known in the art, including silicones. The final parts may be stacked and optionally placed in boxes and palletized.

Another method of forming trays involves natural fiber such as bagasse, bamboo and wheat straw fiber. This involves creating a water-based slurry of the natural fiber called a “pulp”. A mesh screen shaped in the form of the tray is placed in contact with the slurry and vacuum is applied to it. This pulls water through the screen and deposits the fibers on the surface of the screen. The tray is the dried and optionally hot-pressed in a complementary mold prior to removal of the final part. Such processes are known as “molded pulp”, “transfer molded pulp” and “thermoformed fiber”.

### Tray Materials

Multiple classes of materials are suitable for fabricating trays. These compositions include materials disclosed in International Publication No. WO 2013/096531, which is incorporated by reference herein.

In one embodiment, thermoplastic materials are included in the construction of trays used in the process. These thermoplastic compositions can be processed into trays using methods known in the art including but not limited to thermoforming, injection molding and blow molding. These materials may be processable via thermoforming. These compositions include materials disclosed in International Publication No. WO 2013/096531, which is incorporated by reference herein. The materials may be biodegradable and non-phytotoxic. This thermoplastic class of trays includes, but is not limited to: poly(lactic acid), poly(butylene succinate), poly(butylene succinate adipate), poly(butylene terephthalate adipate), poly(ethylene succinate), poly(hydroxybutyrate), poly(hydroxyl valerate), poly(hydroxybutyrate-co-valerate), cellulose acetate, cellulose acetate butyrate, cellulose acetate phthalate, cellulose acetate propionate or poly(caprolactone). These resins may be blended with additives which may serve to accelerate the degradation of the resins. Fillers include, but are not limited to, starch, thermoplastic starch, cellulose, lignocellulosic fiber, bagasse fiber, wood flour, corn stover, calcium carbonate, stearic acid treated calcium carbonate, calcium oxide, kaolin, feldspar, wollastonite, silica, diatomaceous earth, talc, titanium dioxide, clays, strontium hydroxide, barium sulfate, iron oxide, beryllium oxide, glass, alumina, plant-based proteins and keratin. In addition, the materials may contain catalysts to accelerate degradation. Catalysts include, but are not limited to, acids, bases, photosensitizers, oxidation catalysts, metal salts and photoacid generators. Other additives may be present such as antioxidants, plasticizers, lubricants and processing aids.

In one embodiment, the blends of these components are formed by melt compounding using equipment known in the art such as twin screw extruders. Compounding methods include but are not limited to those described in “Mixing and Compounding of Polymers: Theory and Practice” Manas-Zloczower, Ica, 2009.

In another embodiment, a second class of tray materials consists of natural fibers. The natural fibers include, but are not limited to, bagasse fiber, bamboo fiber, wheat straw fiber, kenaf fiber, sisal, hemp, cellulose and lignocellulosic fiber. These compositions may also contain additives to impart moisture resistance and moisture barrier properties. Such additives may include, but are not limited to waxes, tars, emulsion polymers and other hydrophobic substances. A moisture barrier film may be laminated to the tray. Such barrier films may have compositions similar to those described for the thermoplastic class of tray materials.

In another embodiment, a third class of tray materials consists of thermoset resins, including but not limited to urea formaldehyde, melamine formaldehyde, phenol formaldehyde, epoxy resins and composites of these with fibers such as cellulose and natural fibers (bagasse, sisal, kenaf, hemp, etc). These materials may be formed into trays using compression molding or injection molding.

### Storage

After seed assembly there may be a period of storage prior to planting seeds in a field. The length of storage varies and may depend on weather conditions, production, and demand. The range of storage times include about 0-8 weeks, extending up to as much as about 52 weeks. In one embodiment, the seeds are stored in a controlled environment where the plantlets do not grow, yet maintain viability for field planting. This approach comprises use of a controlled environment with temperatures between about 5-15° C. and light between about 0-250 uE.

A second approach uses the storage period to harden and acclimate the plantlets using representative growing conditions. For this approach a growth chamber, greenhouse, or screenhouse could be used where the temperatures range from about 20-35° C. and light ranges between 5 uE to that of natural sunlight. Plantlets may be stored as bare plants or in any of the seed structures included in International Publication No. WO 2013/096531, which is incorporated by reference herein.

Any seed structure listed in International Publication No. WO 2013/096531, or in the tray Examples described herein, for example in Examples 8-10, may also be used for plantlet storage. Regenerable plant tissue that was used to form plantlets after about 1-4 weeks in a bioreactor may also be placed into trays or artificial seed structures and stored under conditions described herein, for example in Examples 6 and 7. A greenhouse or a screenhouse may also be used to harden the plants instead of placing them in a growth chamber under sterile conditions.

Another aspect of the disclosure features a method of storing at least one plantlet placed within a structure, comprising one or more of: a) maintaining a moisture content within said seed structure of between 40%-80%, b) supplying a light source, and c) maintaining a temperature of at least 10° C. to 30° C., wherein the fully formed plants are stored to maintain viability for at least 1 week. The structures may also comprise media that contains hormones and/or hormone inhibitors. The structure may be, but is not limited to, a cavity of an array structure, a tube-like structure, or a support structure.

Another aspect of the disclosure features a method of forming plants from plantlets or regenerable plant tissue before transferring to a field. The method comprises exposing the plantlets to conditions that improve viability of the plants in said field. The conditions include one or more of the following: a) increasing light intensity, b) eliminating added plant hormones, c) reducing humidity, and d) contacting plant roots with media supports such as Rockwool, Metro Mix 360 ®, soil, or other material.

Another embodiment features a method of inserting the fully formed plants into artificial seed structures. The method comprises transferring plants from an array structure to a tray structure. The transfer method is selected from the group comprising, or alternatively consisting of, piston force transfer, vacuum transfer, sieving, manual transfer, and mechanical transfer.

Another aspect features a method of transporting an artificial seed structure, comprising introducing an artificial seed structure into a space having one or more conditions selected from the group comprising, or alternatively consisting of: stacking trays, temperature controlled environments, humidity controlled environments, machinery to transfer artificial seed structures to a planter, machinery to transfer the artificial seed structures to transport vehicles, and transporting said seed structures.

### Planting

In another aspect, the seed structures are transported to a field and planted using mechanical means that ensure viability and vigor of the plants.

One embodiment of this disclosure is that artificial seeds may be planted in the soil in a variety of ways, including but not limited to, planted manually or by mechanization. One example of planting the artificial seed is described in Example 18.

One aspect features a method of planting the plant contained within an artificial seed structure.

Another aspect features a method of planting a crop from excised meristems comprising: a) obtaining the excised meristem to form regenerable plant tissue, b) using the regenerable plant tissue of (a) to form plantlets, c) making the plants from the plantlets of (b), d) placing the plantlets of (b) or the plants of (c) into an artificial seed structure, and e) planting the artificial seed structure of (d), to obtain a crop.

Another aspect features a method of planting sugarcane, said method comprising (i) obtaining a regenerable plant tissue comprising, a) growing excised sugarcane meristem tissue on solid support media in the presence of plant hormones, and b) transferring the tissue from step a) to liquid growth media in the presence of plant growth regulators; and growing to form the regenerable plant tissue; (ii) subdividing said regenerable plant tissue comprising, a) cutting said tissue into smaller segments having a size from about 1 mm³ up to about 1000 mm³, b) placing said cut tissue into temporary immersion with liquid medium for at least about 3-10 days, and c) replacing the medium after about every 3-10 days; (iii) further growing said subdivided regenerable plant tissue, comprising, a) placing the plant tissue into at least one of the following: a cavity of an array structure, a tube-like structure, or a support structure; and b) growing said tissue; (iv) forming plantlets from said regenerable plant tissue by growing in media lacking hormones; (v) transferring the plantlets from the structures of (iii) to an artificial seed; (vi) forming plants from said plantlets, comprising exposing the plantlets to one or more of the following conditions: increased light intensity, reduced humidity, and contact of plant roots with media supports such as Rockwool, MetroMix®360, soil or other material; (vii) transporting said artificial seed structure with said plants, comprising, a) introducing said artificial seed structure into a space, and b) transporting said seed structures to a field; and (viii) planting the artificial seed structure.

Another aspect features a method of planting sugarcane, said method comprising: (i) obtaining a regenerable plant tissue comprising, a) growing excised sugarcane meristem tissue on solid support media in the presence of plant hormones, and b) transferring the tissue from step a) to liquid growth media in the presence of plant growth regulators; and growing to form the regenerable plant tissue; (ii) subdividing said regenerable plant tissue comprising, a) cutting said tissue into smaller segments having a size from about 1 mm³ up to about 1000 mm³, b) placing said cut tissue into temporary immersion with liquid medium for at least about 3-10 days, and c) replacing the medium after about every 3-10 days; (iii) further growing said subdivided regenerable plant tissue in an artificial seed structure; (iv) forming plantlets from said regenerable plant tissue by growing in media lacking hormones; (v) forming plants from said plantlets, comprising exposing the plantlets to one or more of the following conditions: increased light intensity, reduced humidity, and contact of plant roots with media supports such as Rockwool, MetroMix®360, soil or other material; (vi) transporting said artificial seed structure with said plants, comprising, a) introducing said artificial seed structure into a space, and b) transporting said seed structures to a field; and (viii) planting the artificial seed structure.

In one embodiment said planting comprises controlled planting depth.

In another embodiment said planting comprises addition of supplemental nutrients.

In another embodiment said planting comprises separation of individual artificial seeds from tray structures.

In another embodiment said planting comprises cutting of the artificial seed structure.

In another embodiment said planting comprises orienting the artificial seed structure during planting.

In another embodiment said planting comprises addition of degradation promoting agents.

In another embodiment said planting comprises machinery to transfer said artificial seed structures to a planter.

In another embodiment, said planting is in a field.

The excised meristems, the regenerable plant tissue, and the plantlets are selected from the group consisting of sugarcane, a graminaceous plant, Saccharum spp, saccharum spp hybrids, Miscanthus, switchgrass, energycane, sterile grasses, orchid, bamboo, cassava, corn, rice, banana, potato, sweet potato, yam, pineapple, trees, willow, poplar, mulberry, Ficus spp, oil palm, date palm, poaceae, verbena, vanilla, tea, hops, Erianthus spp, intergeneric hybrids of Saccharum, Erianthus and Sorghum spp, African violet, apple, date, fig, guava, mango, maple, plum, pomegranate, papaya, avocado, blackberries, garden strawberry, grapes, canna, citrus, lemon, orange, grapefruit, tangerine, or dayap. In another embodiment, the excised meristems, the regenerable plant tissue, and the plantlets are genetically modified.

Though the disclosure described herein discusses sugarcane, the disclosure is not to be limited by this discussion, and it is contemplated that the methods and compositions disclosed herein are pertinent to other plant types disclosed and known to those of skill in the art.

A trait, as used herein, refers to the phenotype derived from a particular sequence or groups of sequences. For example, herbicide-tolerance polynucleotides may be stacked with any other polynucleotides encoding polypeptides having pesticidal and/or insecticidal activity, such as Bacillus thuringiensis toxic proteins (described in U.S. Pat. Nos. 5,366,892; 5,747,450; 5,737,514; 5,723,756; 5,593,881; Geiser, et al., (1986) Gene 48:109; Lee, et al., (2003) Appl. Environ. Microbiol. 69:4648-4657 (Vip3A); Galitzky, et al., (2001) Acta Crystallogr. D. Biol. Crystallogr. 57:1101-1109 (Cry3Bb1) and Herman, et al., (2004) J. Agric. Food Chem. 52:2726-2734 (CrylF)), lectins (Van Damme, et al., (1994) Plant Mol. Biol. 24:825, pentin (described in U.S. Pat. No. 5,981,722), and the like. The combinations generated can also include multiple copies of any one or more of the polynucleotides of interest.

Polynucleotides encoding polypeptides, alone or stacked with one or more additional insect resistance traits can be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, and the like). A complete agronomic package of improved crop quality with the ability to flexibly and cost effectively control any number of agronomic pests may be generated.

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere, et al., (1987) Meth. Enzymol. 143:277) and particle-accelerated or “gene gun” transformation technology (Klein, et al., (1987) Nature (London) 327:70-73; U.S. Pat. No. 4,945,050, incorporated herein by reference). Additional transformation methods are disclosed below.

Genetically modified plants may have a transgene of interest.

As used herein, an “area of cultivation” comprises any region in which one desires to grow a plant. Such areas of cultivations include, but are not limited to, a field in which a plant is cultivated (such as a crop field, a sod field, a tree field, a managed forest, a field for culturing fruits and vegetables, etc), a greenhouse, a growth chamber, etc.

Unless specified otherwise, for example in a particular experiment, a “control” or “control plant” or “control plant cell” provides a reference point for measuring changes in phenotype of the subject plant or plant cell, and may be any suitable plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell which is genetically identical to the subject plant or plant cell but which is not exposed to the same treatment (e.g., herbicide treatment) as the subject plant or plant cell; (e) the subject plant or plant cell itself, under conditions in which the gene of interest is not expressed or (f) the subject plant or plant cell itself, under conditions in which it has not been exposed to a particular treatment such as, for example, a herbicide or combination of herbicides and/or other chemicals. In some instances, an appropriate control plant or control plant cell may have a different genotype from the subject plant or plant cell but may share the herbicide-sensitive characteristics of the starting material for the genetic alteration(s) which resulted in the subject plant or cell (see, e.g., Green, (1998) Weed Technology 12:474-477; Green and Ulrich, (1993) Weed Science 41:508-516.

Embodiments of the present disclosure are further defined in the following Examples. It should be understood that these Examples are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight; and pressure is at or near atmospheric.

### EXPERIMENTAL

### Example 1

### Meristem Isolation and Culturing

General procedures have been previously outlined (International Publication No. WO 2013/096531, incorporated by reference herein). In summary, sugarcane stalks (approximately 6-9 months old) were grown in the field or greenhouse such that the apical meristem was at least 30 cm above the soil line. Stalks were cut near the base of the stalk. Leaf blades were trimmed leaving the leaf sheaths intact. Stalks were cut into 20 cm long pieces with the apical meristem in the center. One to three outer leaf sheaths were removed and the stalk pieces were submerged in 70% ethanol for 20 minutes. The stalks were moved to a clean container inside a laminar flow hood. A water rinse is optional.

Successive leaf sheaths were removed to expose the meristem. The stalk pieces are further trimmed leaving 1-2 cm margins around the meristems. The meristem section was split in half longitudinally with the cut either separating or bisecting the axillary buds. The two halves were placed cut side down onto proliferation agar media (0.8% agar or 0.43% phytagel containing full strength Murashige-Skoog salts+vitamins (MS) (Phytotech Labs, Overland Park, Kans.)+3% sucrose and 4 mM BAP (6-Benzylaminopurine); 0.8% agar; or 0.43% Phytagel (Sigma-Aldrich, St. Louis, Mo.), in disposable plastic Petri dishes.

Alternatively, the dissection was done on a clean countertop by hand with scalpels or with a mechanical slicer and the half bud segments moved under the laminar flow hood and soaked in 12% Clorox in autoclaved water for 20 minutes, then rinsed in sterile water for 2 minutes, then moved to agar media as above.

Material was incubated at 26° C. in growth chambers or rooms with approximately 30 uE/m2/s of broad spectrum light intensity (PAR). Each ‘half explant’ was trimmed at 7-10 days and placed onto fresh media, this step can be repeated weekly thereafter 0-8 times. Proliferating meristems, also referred to as mericulture tissue, were trimmed and placed into temporary immersion reactors to generate “regenerable plant tissue” and/or mericulture tissue. The timing of any of these steps was variable based on cultivar response.

### Example 2

### Culturing of Regenerable Plant Tissue

General procedures have been previously outlined (International Publication No. WO 2013/096531, incorporated by reference herein). In summary, half-explants were trimmed of excess stalk tissue, leaving behind the proliferating buds areas. Trimmed tissue was placed into a temporary immersion chamber for liquid culture, or back onto solid media. In the case of temporary immersion, the chamber was placed in a growth chamber, or room, with 30 uE/m2/sec of PAR light, and was hooked to tubing connected to pumps to allow standard immersion cycling. Media (MS+3% sucrose+4 mM BAP+3 mg/L paclobutrazol) was changed weekly. Harvest occurred 2-20 weeks after initiation, depending on whether the tissue is grown on solid or liquid media, and the overall mass of the regenerable plant tissue (RPT).

The RPT was subdivided into additional growth chambers or plates as needed. Multiple rounds of growth cycles were possible. No root formation is detected as long as BAP is present in the culture.

### Example 3

### Plantlet Formation

Plantlet formation (production of plants with shoots and roots), also called regeneration, was initiated by transferring the RPT to media without hormones. RPT fragments were made using scalpels, chopping tools, or by large scale dicing of material by mechanical means. Fragment size was determined by end usage: RPT fragments for growth in arrays were approximately 1-10 mm cubes, for growth in temporary immersion chambers somewhat larger fragments 5-20 mm were used, or the RPT was left unfragmented. The media used was MS+3% sucrose. Plantlet formation occurred at 26° C. in 60 uE/m2/sec of PAR light (16 hours on and 8 hours off), and if temporary immersion was used media was cycled over the fragments for 1 minute every 2 hours. Media was replaced weekly and was supplemented with 100 ppm carbenicillin, 0.2% PPM (Plant Preservative Mixture™, Plant Cell Technology Inc. Washington, D.C.), and/or 100 ppm cefotaxime as needed. Supplemental carbon dioxide (1500 ppm) was used in some growth cycles, but is not essential. After 3-4 weeks the MS concentration was decreased to ½ strength to encourage rooting, although not required. The sucrose levels were also decreased to 2% then 1% during this time, although this is not required.

The addition of 5 mM Na silicate to the MS media was made in weeks 3 and 4. There was observational evidence that the addition of silicates improves growth rate and root production in a field test. Silicates accumulated in sugarcane tissues conferring anti-predatory, anti-fungal properties. Silicates were typically added to field sites in order to improve overall growth and field performance.

### Example 4

### Use of Plant Endophytes to Enhance Growth of Micropropagated Sugarcane Plants

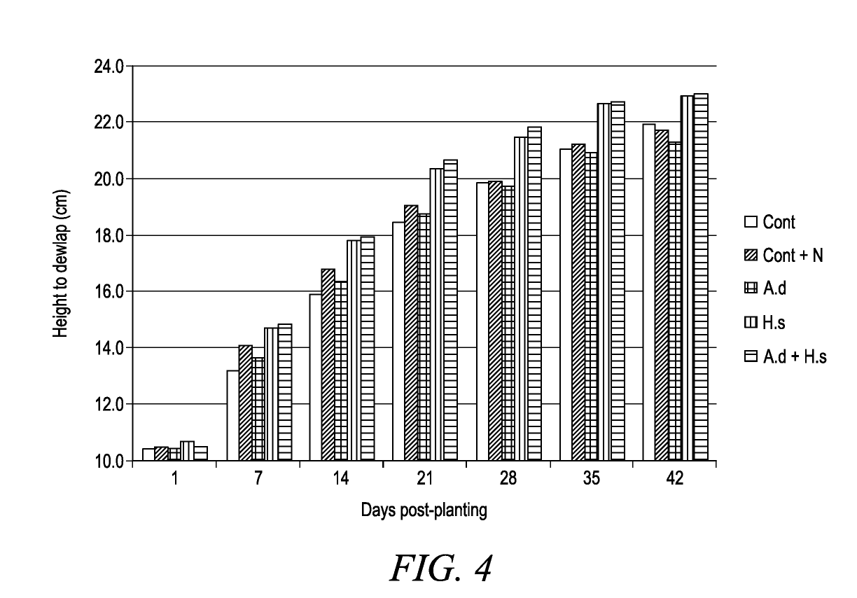

Herbasprillum seropedicae (ATCC#36892) or Glucoacetobacter diazotrophicus (ATCC#49037) cultures, or a mixture of both, were added to micropropagated plants at the end of the in vitro plant regeneration stage. After 24 hrs of inoculation, the plants were removed from in vitro tissue culture and planted in flats containing Matapeake soil (with very low nitrogen levels) to determine if the microbes enhanced plant growth under these conditions. As can be seen in FIG. 4, plants exposed to a mix of both microbes or to just the Herbaspirillum strain did show increased growth. H. seropedicae produced a growth benefit when added to the regenerated plants, while the addition of G. diazotrophicus did not produce additional growth benefit compared to the control. The growth benefit was not a statistically significant change but did suggest an effect.

Enhanced Longer Term Growth of Micropropagated Sugarcane Plants Inoculated with Known Diazotrophic Endophytes

Plants were regenerated from micropropagation, exposed to a mixture of Pantoea agglomerans #100 and Herbaspirillum seropedicae ATCC#35892 in the final day of temporary immersion, and then planted into a mixture of LB2:Turface potting medium. This potting medium contains only trace levels of available nitrogen. Three levels of nitrogen (1.5 mM, 3.0 mM, 6.0 mM); as nitrates in nitrogen minus Hoagland's fertilizer (Epstein, 1972) were used for this experiment. Plants were moved from small pots to larger pots after 6 weeks. In the small pots after six weeks of growth, there was no significant difference in growth rate or total growth observed between inoculated and uninoculated plants. The plants were transferred to larger (12″) pots and continued observations of growth were made to 5 months post planting.

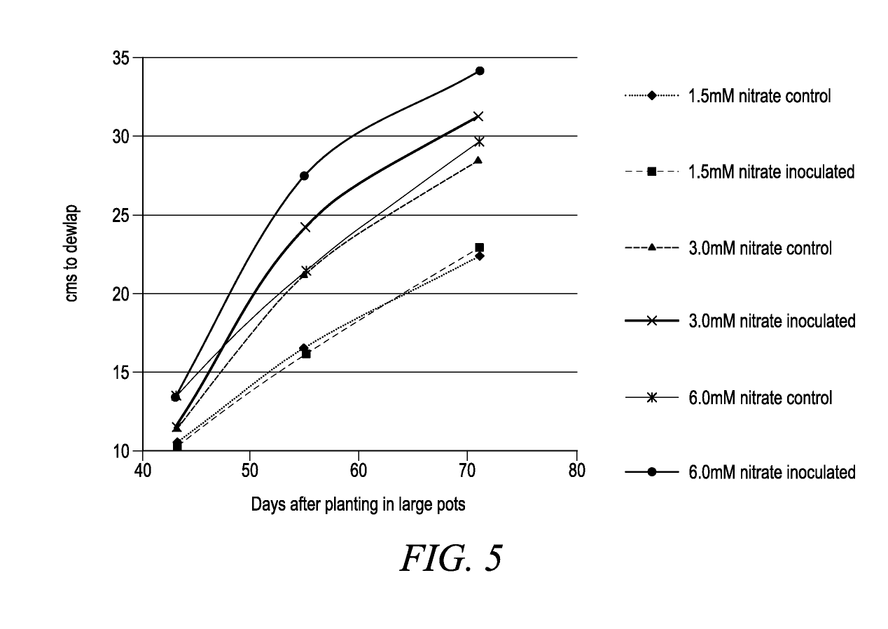

As shown in FIG. 5, two weeks after transplant to larger pots, a statistically significant increase in height was observed in the plants which had been inoculated and fed with either 3.0 mM or 6.0 mM nitrates over respective controls. Mid-way through the study (time point 72 days), a statistically significant increase in growth was shown in the inoculated 6.0 mM nitrate treatment over the control. No statistical differences were seen between the inoculated and uninoculated control with 1.5 mM nitrate treatment throughout the experiment. All 3.0 mM and 6.0 mM nitrate treated plants eventually reached the same height and then leveled off.

In this example, plants receiving limited soluble nitrate (1.5 mM) were not visibly affected by the addition of endophytic cultures. However, in plants given sufficient nutrition (3.0 mM or 6.0 mM nitrate), the positive effects of microbe inoculation became apparent after longer term growth (72 days).

### Example 5

### Array Structures for Regenerable Plant Tissue Proliferation and Plantlet Singulation

The array in this experiment was a ½″ thick block consisting of several ¼″, ⅜″, and ½″ diameter holes and was rapid prototyped from a 3D printer. It was made of polycarbonate, although other suitable and autoclavable materials may include polypropylene, polypropylene copolymer, polymethylpentene, polytetrafluoroethylene (PTFE or Teflon), fluorinated ethylene propylene, perfluoroalkoxy (PFA), and polysulfone. One or more fragments of regenerable tissue were placed into each hole in the array. The array was loaded with fragments under sterile conditions and placed in a bioreactor where it underwent four weeks of regeneration. The results of this experiment are shown in the table below:

TABLE 1

| | No. of | Final | No. of Viable | No. of Plantlet |
|---|---|---|---|---|
| Array Cavity | Holes | mass [g] | Plantlets | Shoots per Hole |
| ½″ OD hole | 30 | 25.60 | 29 | 5+ |
| ⅜″ OD hole | 38 | 17.31 | 29 | 3+ |
| ¼″ OD hole | 54 | 4.54 | 16 | 1.5 |

Total Initial Mass [g]: 8.00
Total Final Mass [g]: 47.45
Mass Fold Increase [—]: 5.93

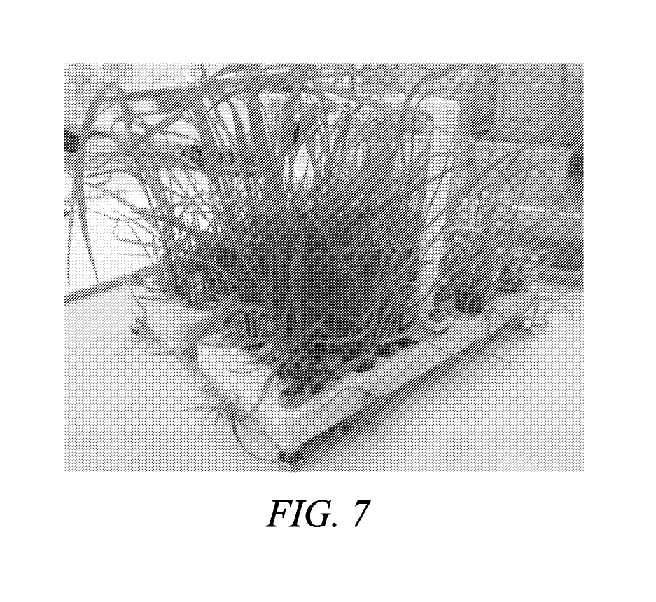

FIG. 6 shows the arrangement of this array. FIG. 7 shows the resultant sugarcane plantlets regenerated after 4 weeks. Plantlets were generated from each of the hole sizes with percentage of viable plants proportionate to hole size.

In another experiment, fragments were loaded into a 1,000 cavity array in a larger 20 L volume bioreactor. This array was made from polysulfone although any other autoclavable material may be used. The array was fabricated from a ½″ thick block of material and the cavities were formed by drilling. In this experiment, hole size was ⅜″ to maximize viability. Fragments of regenerable tissue were placed into and over the cavities of the array. Media was pumped into the bioreactor completely flooding the cavities and allowing the fragments to migrate and disperse over the array. After this first immersion cycle, adjustments were made so as to not flood the cavities during the rest of the experiment. This experiment was conducted for 6 weeks and the results were: Initial Tissue Mass [g]=64; Final Tissue Mass [g]=1643; Mass Fold Increase [−]=25.6. Of the plantlets recovered from half the array, or 500 cavities, there were 206 plantlets between 5 and 10 cm in length; 282 plantlets larger than 10 cm in length; and there was a 98% regeneration rate of viable plantlets.

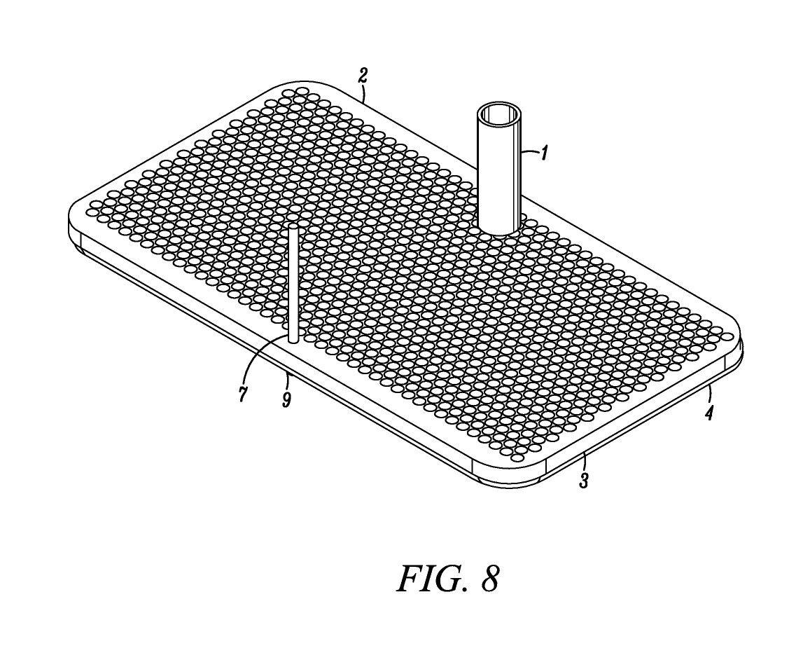

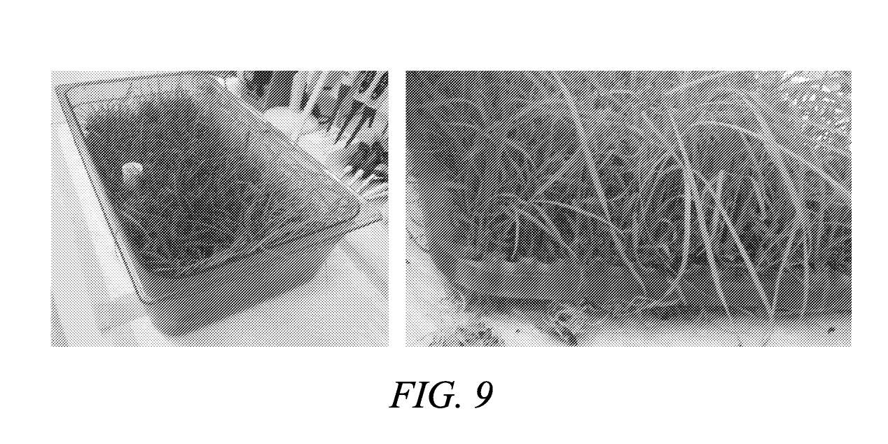

FIG. 8 shows the arrangement of this array. FIG. 9 shows the resultant sugarcane plantlets regenerated after four weeks.

In another experiment a thin film was used to support the fragments. This film had narrow slits cut at the same pitch as the cavities in the array. These slits were in a cross or “X” pattern over the center of each cavity allowing for media to pass through while supporting the fragments above the media. The slits were created using a sharp blade to pierce the film. Other ways to prepare a film in this manner include, but are not limited to: slits, small holes or perforations, any multi-sided opening or a film, paper, etc. with a suitable porosity without any macroscopic openings.

In this experiment, fragments were able to regenerate into viable plantlets on the film. Film materials may be polypropylene, polysulfone, polycarbonate or other autoclavable materials.

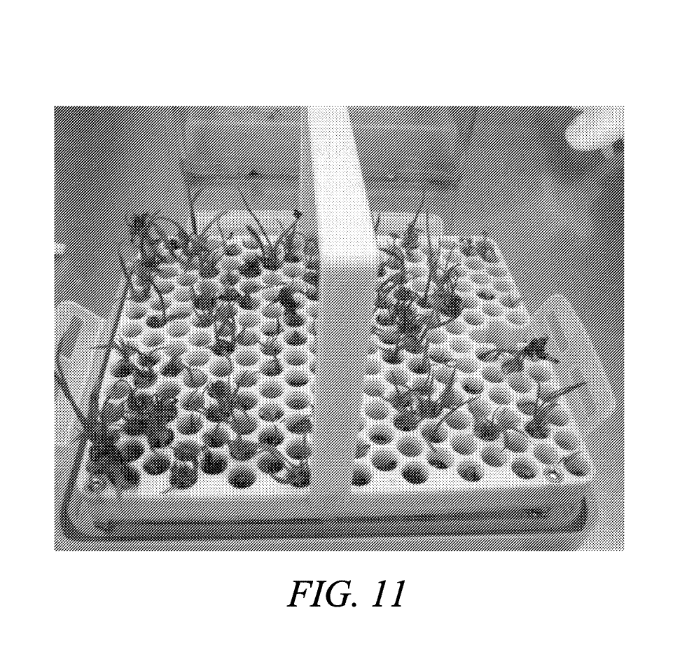

FIG. 10 shows the arrangement of this array. FIG. 11 shows the plantlets regenerating after one week of the four week experiment.

### Example 6

### Regeneration of Meristem Tissue Using Arrays

The bioreactor arrays described in Example 5 were used to generate plants with roots and shoots for use in artificial seed structures (structures as described in International Publication No. WO 2013/096531). The physiological equivalence of these array plants was compared to plants generated using bioreactors without array structures.

Proliferated meristem tissue derived from sugarcane apical meristem explants was fragmented using scalpels to produce 3-5 mm cubes of heterogeneous tissue. Tissue from either Stage 2.1 or Stage 2.2 was used for generation of fragments (see, for example, FIG. 1). If using Stage 2.1 material, the leafy outer layers of proliferated tissue was trimmed away and the ‘bud’ tissue containing multiple meristems was fragmented. From Stage 2.2, trimming of leafy tissue was not done, and fragmentation was less labor intensive due to the friable nature of the tissue.

Fragments of tissue appearing to have meristem potential based on observation of size and density and general health, were used to fill the array holes for each bioreactor by hand. Up to 20 g of fragmented tissue was required to assure filling of each array hole. Some holes could have more than one fragment. Some fragments were lost during immersion by floating out of the holes, some fragments failed to develop into plants of sufficient size for use in synthetic seed constructs (>2 cm from base to dewlap). In 27 trials, an average of 73% of the fragments in array holes developed into usable plants with an average of 8.8 plants/gram of input tissue. Since each hole can contain multiple fragments and each fragment can contain multiple meristems, and these are used as a unit without singulation, each ‘arrayed’ plant can have more total shoot/root units then with standard bioreactor/singulation derived plants. Survival and morphological development of the array derived plants was compared.

Fragments of similar size and source were used in standard bioreactors to compare to array derived plants for health and use in artificial seed constructs. In 10 field studies using standard artificial seed structures (15 ml conical tubes described in International Publication No. WO 2013/096531) containing array derived plants had an average of 75% survival. This was comparable to survival rates of plants derived from bioreactors without array.

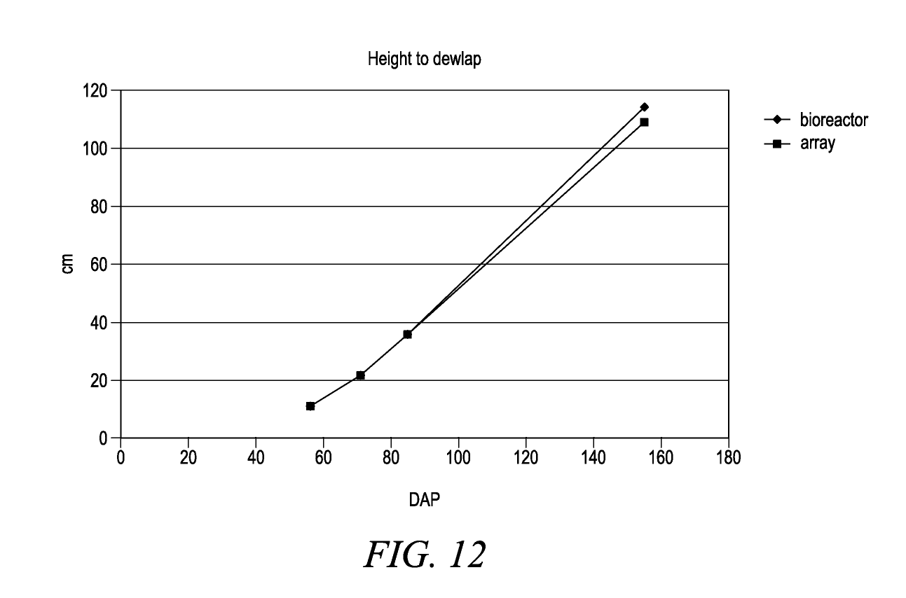

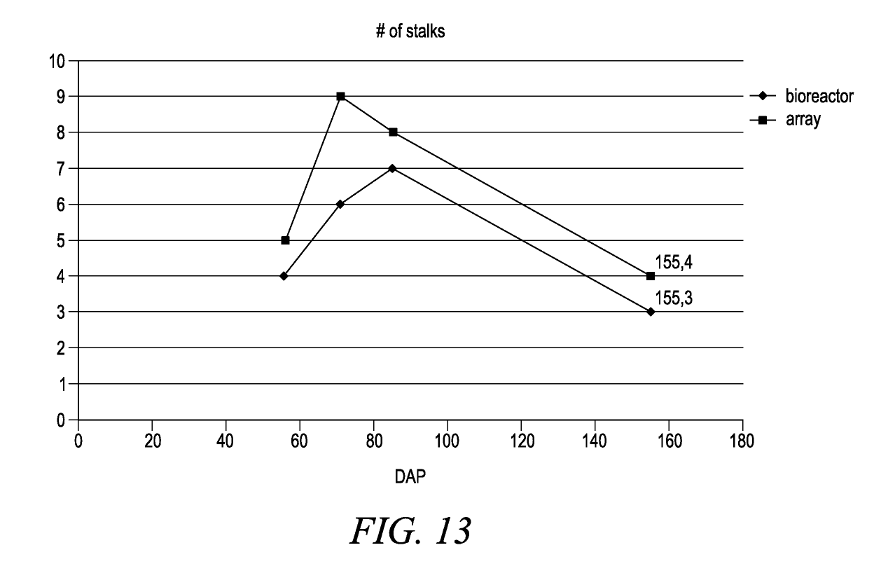

In a direct greenhouse comparison of plants derived from bioreactors with and without arrays (50 plants per bioreactor type), there was no statistical difference in survival in standard artificial seed structures (15 ml conical tubes described in US International Publication No. WO 2013/096531) 150 days after transplanting. Length to the dewlap and number of stalks per ‘plant’ unit were measured. FIGS. 12 and 13 demonstrate that plants derived by either process are statistically similar in morphology and growth rate. Arraying fragments, as described in Example 6, improves the mechanical process of transferring plants into seed structures using mechanical means.

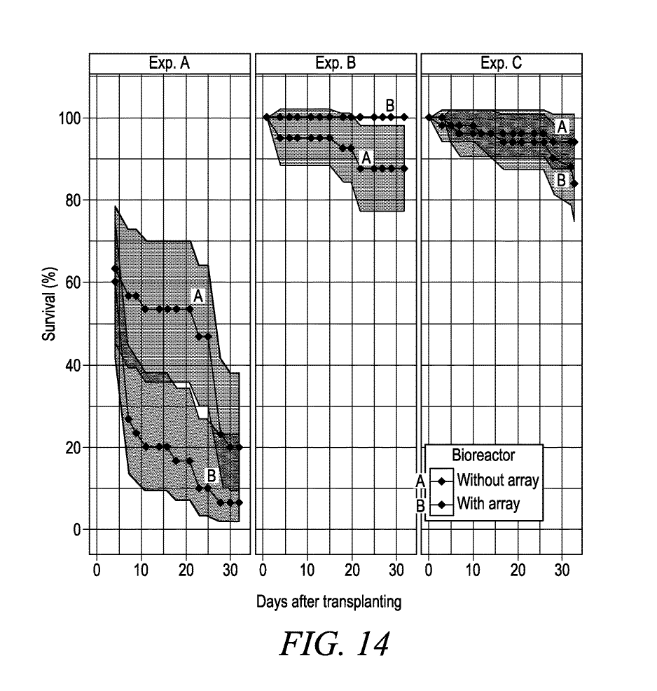

In three field experiments, plants coming from conventional bioreactors were larger than plants coming from arrays (Table 2), although for experiment C the difference was not statistically different at the 95% probability level. The three experiments (Exp. A-C) varied in terms of field environmental condition, as evident from the average survival (FIG. 14) between planting time and 30 days later. Nevertheless, survival of plants in standard artificial seed structures (15 ml conical tubes described in International Publication No. WO 2013/096531) varied less than 15% between the types of bioreactor irrespective of the testing environment. Survival was poor in one case (<20%, Exp. A) and very good in the other two (>80%, Exp B and C). In two of the experiments (A and C), survival was better for the plants grown in bioreactors without the array. The contrary was true in experiment B.

TABLE 2
Shoot length after regeneration

| | Experiment | | |
|---|---|---|---|
| | A | B | C |
| | cm to the dewlap | | |
| Conventional | 2.87 a | 4.70 a | 4.04 a |
| Array | 2.50 b | 2.70 b | 3.86 a |

Figures followed by different letters in the same column are significantly different from each other at the 95% probability level.

### Example 6A

### Regeneration of Fragmented Tissue on Rockwool

Fragmented regenerable sugarcane tissue will regenerate into plantlets on a substrate of solid Rockwool or loose Rockwool without media changes in a static bioreactor. Regenerants continue to grow when transferred to soil as long as they have developed leaves elongated greater than 2 cm.

The addition of one week temporary immersion after fragmentation is not necessary but increases the chance of developing regenerants that produce leaves greater than 2 cm during static incubation. In addition, experiments performed have shown that plantlets are produced when using static incubation on three types of Rockwool substrate; thick solid (3 cm) thin solid (1.5 cm) or thin (1.5 cm) loose. Three sizes of initial regenerable tissue fragments have been tested; small, which are cubes of 1 to 3 mm, medium, which are cubes of 3 to 6 mm and large, which are cubes of 6 to 10 mm. All 3 sizes also produce plantlets.

Proliferated bud material (stage 2.2 material) was fragmented by hand into cubes of 1 to 3 mm (small) and 3 to 6 mm (medium) in a biohood using sterile technique. Ten grams of small and medium fragmented tissue was added individually to 3 Temporary immersion bioreactors and incubated at 26° C., on a 16 hour day/8 hour night photoperiod with 30 uE/m2/s photosynthetically active radiation from fluorescent tubes during the day and no light during the night using a standard immersion (1 min/2 hrs) protocol in 300 ml of regeneration media (½ MS, 1% sucrose, 0.2% PPM (Plant Preservative Mixture™, Plant Cell Technology Inc. Washington, D.C.)) was used.

After 1 week in the RITA bioreactor, the tissue was removed and then transferred to a sterile Plant Form bioreactor with Rockwool covering the entire base. Single fragments of both sizes were added to each of three Plant Form bioreactors. One Plant Form bioreactor having solid, thick Rockwool 3 cm thick with 1 L of MS+3% sucrose, one having thin, solid Rockwool 1.5 cm thick with 500 ml of MS+3% sucrose and one having a layer of loose Rockwool 1.5 cm thick (30 grams of dry, loose material) with 500 ml of MS+3% sucrose. Openings to the Plant Form bioreactor were fitted with 0.2 μm filter disks to maintain sterility during a 9 day incubation at 26° C., on a 16 hour day/8 hour night photoperiod with 30 uE/m2/s photosynthetically active radiation from fluorescent tubes during the day and no light during the night but no media exchanges were made during 9 days of incubation.

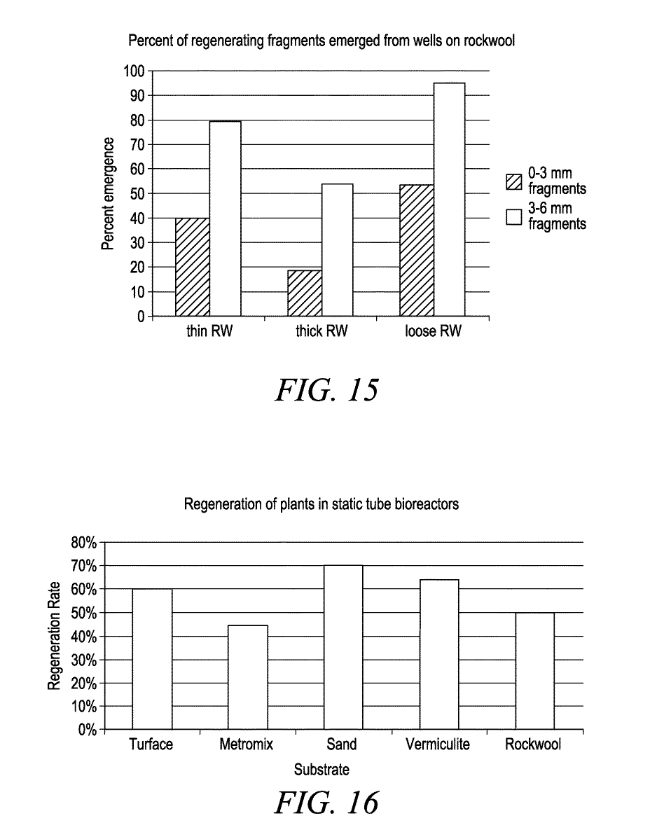

On the 9^{th} day of static incubation the sucrose was rinsed from each Plant Form bioreactor using 3 liters of non-sterile water then moved for an additional 13 days to a growth chamber at 26° C., on a 16 hour day/8 hour night photoperiod with 300 uE/m2/s photosynthetically active radiation (PAR) from fluorescent tubes and incandescent bulbs during the day and no light during the night. The lids of the Plant Form bioreactors were initially left on, but were gradually removed over the course of the next 7 days to allow the plants to gradually adapt to reduced atmospheric humidity. Plants were then maintained under these conditions for an additional 14 days with occasional irrigation to prevent desiccation. Under these conditions, 18-50% of the small fragments and 50-90% of the medium fragments regenerated into plants (FIG. 15).

### Example 6B

### Regeneration of Fragmented Material within Artificial Seeds Inside Static Bioreactors

Fragmented regenerable sugarcane tissue will regenerate into plantlets on a variety of solid substrates saturated with tissue culture media without media changes in a static bioreactor. Regenerants continue to grow when transferred out of the sterile static bioreactor into non-sterile conditions requiring autotrophic growth. The solid substrates can be contained in a structure designed for planting in the field, which simplifies the production process by eliminating the necessity of transferring the regenerated plants from an intermediate container used for the bioreactor stage into the ultimate planting structure.

Solid substrates, which all supported plant regeneration, included sand, Metromix® 360, Turface, Rockwool, and vermiculite. Substrates were placed within cylindrical tubes (11 mm diameter×140 mm in length) with the substrate filling about 15-90% of the length of the tube. Each substrate was saturated with standard nutrient media used for plant regeneration (MS+3% sucrose, supplemented with 100 ppm carbenicillin, 0.2% PPM™ (Plant Preservative Mixture™, Plant Cell Technology Inc. Washington, D.C.), and 100 ppm cefotaxime as necessary) Plantlet formation occurred at 26° C. in 25-100 μmol/m²/sec of PAR light (16 hours on and 8 hours off), with no nutrient exchanges during the regeneration period.

Proliferated bud material (stage 2.2 material) was fragmented using a scalpel into cubes of about 3 mm in a biohood using sterile technique. Substrates (sand, Metromix®360, Turface, Rockwool, and vermiculite) were sterilized by autoclaving. One end of each tube was stapled to roughly close, but not seal the tube, in order to contain the substrate. Tubes were then sterilized by immersion in 100% ethanol for 30 minutes followed by allowing them to drain and air dry within a biohood. Each tube was then filled to 50-90% of its height with a sterile solid substrate. Sterile nutrient medium (MS+3% sucrose, supplemented with 100 ppm carbenicillin, 0.2% PPM (Plant Preservative Mixture™, Plant Cell Technology Inc. Washington, D.C.), 100 ppm cefotaxime) at ˜2-5 ml/tube was added to each tube to saturate the substrate. For Turface samples, the tubes were then placed in a container of sterile medium, such that the height of the medium in the container was about 1 cm below the height of the substrate in the tube, in order to ensure that the substrate remained saturated with medium. One 3 mm tissue fragment was added to the top of the substrate in each tube. All tubes were then placed inside closed, sterile, clear containers (18×15×15 cm). Containers had 5 small openings (4 mm in diameter) containing 0.2 μm air filters which allow a small amount of passive gas exchange with the outside atmosphere. Otherwise, the containers were kept entirely closed during incubation for the next 1-3 weeks and no media exchanges were made.

After 1-3 weeks of incubation, the tubes were removed from the containers and ˜10 ml of non-sterile deionized water was added to the top of each tube and allowed to drip out the bottom to wash out excess media. Tubes were then transferred to non-sterile conditions in a controlled environment plant growth chamber (25° C. in 250 μmol/m²/sec of PAR light (16 hours on and 8 hours off), at about 100% relative humidity and the plants were allowed to continue to grow for 1-3 additional weeks.

Results showed that fragments regenerated into plants and subsequently grew in non-sterile conditions at high rates on all 5 substrates. Light levels ranging from 25 to 100 μmol/m²/sec during the sterile bioreactor stage did not affect regeneration rates. Overall, 41-77% of the fragments regenerated into plants within 2 weeks under these conditions. FIG. 16 shows representative results for one particular set, regenerated for 1 week in the static bioreactor at 46 μmol/m²/sec and then transferred to a non-sterile growth chamber for an additional 1 week. Under these conditions, depending on the substrate, 44-70% of the fragments regenerated into plants.

### Example 7

### Storage and Hardening of Regenerable Plant Tissue and Plantlets

The effect of time, temperature, and light on storage of plantlets was studied, in combination with the effect of chemical treatment. Seed structures consisted of 4″ Starcla™ tubes. Tubes were extruded from a resin, such as a blend of polybutylene succinate, polylactic acid, and starch, using a 1½″ Davis Standard-extruder. Tubing was made with ⅝″ I.D. and cut into 4″ length tubes. The seeds were assembled by heating the bottom of the tube and pressing into a crescent shape where there is 2-3 millimeter gap between both sides. Two grams of potting media were added to each tube. One sugarcane plantlet was placed inside the tube structure ensuring the roots were placed in the soil and the shoot remained in the airspace. Two milliliters of water were added to the structure and then the top was prepared by heating and forming a crescent shape with a 2-3 millimeter gap between both sides.

Two types of chemical treatments were used during regeneration. The first treatment was paclobutrazol (paclo), which was introduced into the media for weeks 3 and 4 of plantlet formation. The solution contained 2% sucrose, ½ strength MS, 100 ppm cefotaxime, and 0.75 mg/L paclobutrazol. The second treatment was abscisic acid (ABA) which was introduced the day before the plantlets were removed from regeneration. The ABA treatment solution contained 1 mg/L ABA, 10 g/L sucrose, and MS solution.

The tubes were stored in partially closed zip bags with 25 tubes per unit. The tubes were stored according to conditions shown in Table 3. After storage the seeds were planted. No irrigation was used during the time the plantlets were growing in the field. The survival rates of the plantlets were measured after 5 weeks of growth.

TABLE 3

| | Time | | | | |
|---|---|---|---|---|---|
| Condition | (weeks) | Temperature | Treatment | Light | Survival |
| A | 8 | 10 | Paclo | 0 | 0% |
| B | 8 | 30 | Paclo | 200 | 88% |
| C | 8 | 10 | ABA | 5 | 0% |
| D | 8 | 30 | ABA | 0 | 0% |
| E | 4 | 10 | Paclo | 5 | 0% |
| F | 4 | 30 | Paclo | 0 | 0% |
| G | 4 | 10 | ABA | 0 | 0% |
| H | 4 | 30 | ABA | 200 | 50% |
| J | 2 | 10 | Paclo | 0 | 0% |
| K | 2 | 30 | Paclo | 200 | 64% |
| L | 2 | 10 | ABA | 5 | 0% |
| M | 2 | 30 | ABA | 0 | 0% |
| N | 0 | — | Paclo | — | 16% |
| O | 0 | — | ABA | — | 12% |
| P | 0 | — | — | — | 12% |

The best storage conditions were B, H, and K. All of these storage conditions were at 30° C. with 220 uE of light. The paclo treatment had a higher survival rate than the ABA treatment. The controls which were not stored performed worse than the stored seeds indicating a hardening affect under storage at higher temperatures and higher light.

In another experiment the plantlets were stored in array structures rather than tubes. The plantlets used for seed-array assembly were regenerated for four weeks. In this experiment, plantlets were compared that were regenerated for 2 weeks compared to those regenerated for 4 weeks. After the seeds were assembled the plantlets were stored and then planted in potting soil. The time the plantlets were in each stage of the experiment is shown in Table 4.

TABLE 4

| | Regeneration | Storage | Growth |
|---|---|---|---|
| Array 1 | 2 weeks | 2 weeks | 4 weeks |
| Array 2 | 2 weeks | 4 weeks | 4 weeks |
| Array 3 | 4 weeks | 0 weeks | 4 weeks |
| Array 4 | 4 weeks | 2 weeks | 4 weeks |

Regeneration refers to the time spent in temporary immersion array bioreactors. Storage refers to the time assembled seed arrays were stored in the growth chamber (at 31° C. during the day, at 22° C. during the night, 60% relative humidity, and a 13 hr photoperiod (220 uE/m²)). Growth refers to the period after which the seed was planted in a flower pot containing potting media and grown into a plant in the growth chamber.

To assemble the seed arrays, one tray containing 200 wells was filled with potting soil. Each well is 1″×1″×2″ and tapers down to ½″×½″. The tray was placed on a flat tray of water to saturate the soil. One plantlet was placed in each well and then the tray was cut into individual pieces. Another empty tray was cut into individual pieces for the top portion of the seed structure. The top portion was attached to the bottom portion using a hot glue gun. The seeds from arrays 1 and 2 were placed on a flat tray containing water for storage. The seeds from array 4 were placed in a zip lock bag with some additional water. After storage the seeds were planted in a flower pot containing potting media and were watered 3 times a week. The plantlets were monitored for viability at all stages of the experiment. The survival rates during each stage of the experiment are shown in Table 5. This demonstrated that plantlets that were regenerated for 2 weeks were as viable as plantlets that were regenerated for 4 weeks.

TABLE 5

| | Regeneration | Storage | Growth |
|---|---|---|---|
| Array 1 | 91% | 85% | 100% |
| Array 2 | 96% | 96% | 100% |
| Array 3 | 98% | — | 100% |
| Array 4 | 96% | 100% | 100% |

Any seed structure listed in International Publication No. WO 2013/096531, or in the tray examples below, can be used for plantlet storage. Regenerable plant tissue that was used to form plantlets after 1-4 weeks in a bioreactor could also be placed into trays or artificial seed structures and stored under conditions described above. One could also use a greenhouse or a screenhouse to harden the plants instead of placing them in a growth chamber.

### Example 8

### Ultrasonic Welding of Trays

Methods for assembling the seed arrays described in Example 7, or previous examples, using ultrasonic welding were evaluated. The trays that were tested included 200 well trays made from PETG and 12 well trays made from PLA (poly(lactic acid), PLA blends, and PBS (poly butyl succinate). A bottom tray with the openings of the wells facing up was filled with soil to within ⅛″ of the top of the well opening. 2 inch tall plants were manually inserted into the soil, roughly in the center of each well. A second tray with the same well size and spacing was placed upside down over the bottom tray and arranged such that the wells were aligned. This assembly was placed into the ultrasonic welder. A Dukane Model 220 was used to weld the trays together. The settings were:
Frequency: 20 khz
Time: 3.5 sec
Hold: 0.5 sec
Pressure: 70 psi
Anvil: 0.18″ wide×6″ long, steel
Horn: 0.125″ wide×1.5″ long, aluminum
   The assembled trays were placed into the welder with the flat sections of the tray supported by the anvil. The assembled trays were moved along the anvil until the width of the horn covered one side of a well completely. When the welder was actuated the horn lowered into contact with the trays and formed and melted the top and bottom trays together, forming a seal along one side of the well. This operation was repeated 4 times around the well to create a fully sealed structure around the plant. This operation was repeated for each well. The wells were then separated using scissors to cut the web connecting the wells.

In a variation of the above process the anvil was modified so that instead of a flat surface there was a small protrusion, 0.040″ tall by 0.040″ wide in the center of the anvil running the entire length. During the ultrasonic welding process this ridge thins out the connecting web and allows for easier separation of the welded wells.

### Example 9

### Storage of Assembled Seeds

The effects of time, temperature, light, and seed structure during storage on the survival of seeds after storage were evaluated. For this experiment, the seed structure consisted of a 5″ poly(lactic acid) tube. Tubes were extruded from poly(D,L-lactic acid) using a 1½″ extruder. Tubing was made with ⅝″ I.D. and cut into 5″ length tubes. Two seed designs were implemented during the storage experiment, a closed structure where both ends were sealed using heat and an open structure where gas could easily exchange in and out of the tube. For the closed structure, the bottom of the tube was heated and then pressed together forming a seal. Two grams of potting media were added to the tube. One sugarcane plantlet was placed inside the tube structure ensuring the roots were placed in the soil and the shoot remained in the airspace. Two milliliters of water were added to the structure and then the top was sealed by heating and pressing together. For the open structure, the bottom of the tube was heated and pressed into a crescent shape where there is 2-3 millimeter gap between both sides. Two grams of potting media were added to the tube. One sugarcane plantlet was placed inside the tube structure ensuring the roots were placed in the soil and the shoot remains in the airspace. Two milliliters of water were added to the structure and then the top was prepared by heating and forming a crescent shape with a 2-3 millimeter gap between both sides.

Once assembled, the seeds were stored in partially closed zip bags with 25 seeds per unit. The seeds were stored according to the table below and 50 seeds were used per configuration. After storage, the seeds were planted in flowerboxes (60 cm length, 20 cm width, 12 cm height) containing a 50:50 blend of soil and sand. Five percent potting soil was incorporated into the soil to provide organic matter. One bag of seeds (25) was planted in each flowerbox and the seeds were planted so the soil level in the structure aligned with the soil level in the flowerbox. The flowerboxes containing the planted seeds were placed in a growth chamber at 31° C. during the day, at 22° C. during the night, 60% relative humidity, and a 13 hr photoperiod (220 uE/m²)) and watered 1 L of water upon planting. The plants were grown for four weeks and given 1 L of water per flowerbox, three times a week. After four weeks of growth, the survival rate and height of the plants was recorded.

The survival rate and average plant height are shown in Table 6. Storage configurations 5 and 7 produced the best plant survival while storage condition 4 produced no viable plants. This suggests that light and lower temperature were the best conditions for storage of plants produced in this manner.

TABLE 6

| | | | | | | Average |
|---|---|---|---|---|---|---|
| | Temper- | | | | | Plant |
| Config- | ature | Time | Light | | Survival | Height |
| uration | (° C.) | (weeks) | (uE) | Structure | (%) | (cm) |
| 1 | 10 | 2 | 0 | Closed | 82 | 36.1 ± 9.2 |
| 2 | 20 | 2 | 0 | Open | 63.5 | 26.7 ± 7.9 |
| 3 | 10 | 4 | 0 | Open | 57.5 | 30.7 ± 10.9 |
| 4 | 20 | 4 | 0 | Closed | 0 | 0.0 ± 0.0 |
| 5 | 10 | 2 | 5 | Open | 98 | 31.0 ± 10.5 |
| 6 | 20 | 2 | 5 | Closed | 68 | 31.7 ± 9.9 |
| 7 | 10 | 4 | 5 | Closed | 93 | 28.2 ± 9.3 |
| 8 | 20 | 4 | 5 | Open | 34 | 33.2 ± 4.8 |

### Example 10

### Tray Structures

### Blend Compositions

Poly(lactic acid) (Ingeo™ 2003D, Natureworks LLC, Blair, Nebraska), poly(butylene succinate) (Bionolle™ 1001 and 1903, Showa Denko, Japan) and poly(butylene succinate adipate) (Bionolle™ 3020, Showa Denko, Japan), were dried at 50° C. under partial vacuum with a stream of nitrogen in a vacuum oven for 18 h. Starch was dried at 100° C. under partial vacuum with a stream of nitrogen in a vacuum oven for 18 h. Bagasse fiber was dried at 120° C. under partial vacuum with a stream of nitrogen in a vacuum oven for 72 h. The compositions listed in Table 7 were manually blended in polyethylene bags and then compounded using a Prism 16 mm twin screw extruder with barrel and die temperatures set at 180° C. and 150-200 rpm screw speed. The molten strand was quenched in room temperature water and pelletized using a rotary cutter.

TABLE 7
Thermoformable Biodegradable Material Compositions

| Composition | Base Resin | Resin 2 | Additive 1 | Additive 2 |
|---|---|---|---|---|
| 1 | Ingeo ™2003D, | | Iron (III) | None |
| | 99 wt % | | stearate, 1 wt % | |
| 2 | Bionolle ™ | Bionolle ™ | None | None |
| | 1001, 90 wt % | ™1903, 10 wt % | | |
| 3 | Bionolle ™ | Bionolle ™ | Omyacarb ® | None |
| | 1001, 32.4 wt % | 1903, 3.6 wt % | 2SST (CaCO₃), | |
| | | | 64 wt % | |
| 4 | Ingeo ™- | | Corn starch, 20 wt % | None |
| | 2003D, 80 wt % | | | |
| 5 | Ingeo ™- | | Corn starch, 30 wt % | None |
| | 2003D, 70 wt % | | | |
| 6 | Bionolle ™ | Bionolle ™ | Milled bagasse | None |
| | 1001, 76.5 wt % | 1903, 8.5 wt % | fiber, 15 wt % | |
| 7 | Ingeo ™- | | Corn starch, | Oleic acid, |
| | 2003D, 67.5 wt % | | 28.9 wt % | 3.6 wt % |

The compounded pellets of the above compositions were dried at 50-70° C. under partial vacuum with a stream of nitrogen in a vacuum oven for 18 h. The pellets were then compression molded at 190-200° C. between sheets of poly(tetrafluoroethylene) using 375-750 um thick steel shims with a 10 cm square window. The sheets were rapidly cooled with dry ice after pressing and removed from the molds after hardening.

### Degradation Example

150 um thick films of composition 1 from Table 7 were formed via compression molding at 200 C. These were placed in a Q-Labs QUV weatherometer at 50° C. and 0.68 W/m² intensity using 340 nm UVA bulbs for 250 hours of continuous exposure. After exposure, the films had cracked and embrittled to the point of being too weak to manually handle without fracture. By comparison, a control film consisting of Ingeo™ 2003 D with no additives showed no cracking or obvious degradation during a weatherometer exposure experiment with the same conditions.

Thermoforming:

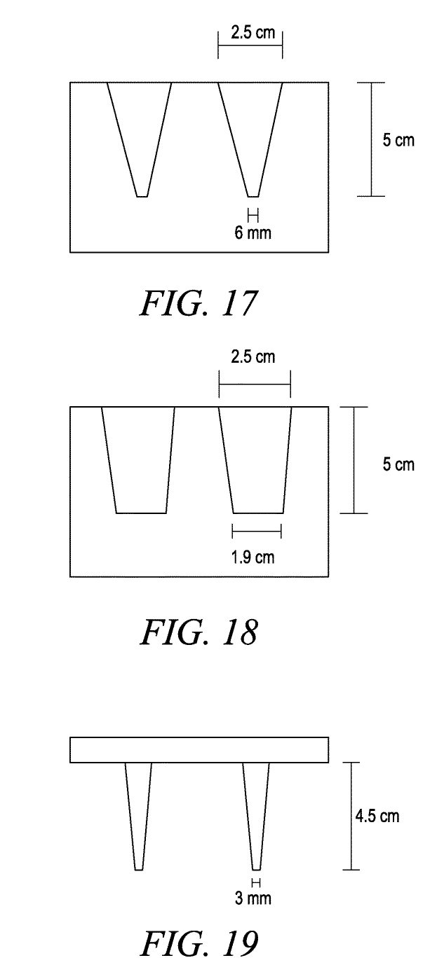

4-cavity top and bottom tray molds were fabricated using rapid prototyping of polycarbonate resin. The mold geometries are shown in FIGS. 17 and 18. Complementary plug assists were also fabricated out of polycarbonate through rapid prototyping and are shown in FIG. 19.

375-1250 um thick 10 cm square sheets of compositions 1-7 from Table 7 were heated to 120-155° C. in a press for 3 min between polytetrafluoroethylene sheets. The sheets were then manually transferred to the mold surface in FIG. 17. The plug assist shown in FIG. 19 was used to deform the sheet to the depth of the mold, and then vacuum was applied to expand the sheet to fill the mold. The part was allowed to cool and then was removed from the mold.

Specifically, 450 um thick sheets of composition 5 were heated to 155° C. for 3 min between polytetrafluoroethylene sheets. The heated, softened sheet was manually transferred to the mold surface in FIG. 17. The plug assist shown in FIG. 17 was used to deform the sheet to the depth of the mold, and then vacuum was applied to expand the sheet to fill the mold. The part was allowed to cool and then was removed from the mold. The resultant part reproduced the mold geometry well and exhibited good rigidity when cooled.

### Example 11

### Tray Based Seed Testing

Non-degradable thermoformed trays made of PET poly(ethylene terephthalate) were obtained from TO Plastics (Clearwater, Minn.). The trays had dimensions of 28×54 cm with 200 wells spaced evenly in a square grid pattern (10×20). Each well had a depth of 5.3 cm and a square open top profile with a width of 2.3 cm. The wells were trapezoidal shaped, with a flat bottom 1.5 cm wide. The bottom of each well had a 6 mm hole in the center. Indented vertical ribs were present along each of the walls of the cell, which were about 1 mm deep and 3 mm wide. The thickness of the sheet prior to thermoforming was 1125 um. The trays wells were separated by cutting them apart manually. A mixture of Metro-Mix® 360 (5.5 g) and deionized water (5.5 g) was added to one well. A sugarcane plantlet, which had been propagated using the herein described tissue culture process, was inserted into the wet Metro-Mix® 360 substrate using forceps. A second tray well was placed over the first and the two halves were hot glued together. A series of modified wells was created by placing the tip of a 50 mL pipette inside a well and through the hole at the bottom, and then heating the well using a hot air gun. The heated well was then pressed against the pipette surface, creating a conical shaped well. A set of seeds was assembled as described above using the conical shaped well for the top of the seed and the undeformed square shaped well for the bottom. A third set of seeds was made using 15 mL conical centrifuge tubes similarly to that described in Example 20, International Publication No. WO 2013/096531. The tip of the conical section of a 15 mL centrifuge tube was cut off, producing a 5-7 mm hole in the top. The entire conical section of the 15 mL tubes was cut off, and the plastic caps removed. Parafilm@ M was manually stretched over the threaded end of the tube, and 4 g Metro-Mix® 360 were added to the tube. A sugarcane plantlet was manually inserted into the soil using forceps and 4 mL deionized water was added to each well. The conical section was re-attached to the main tube by wrapping Parafilm@ against the joint. All three sets of seeds were planted in Metro-Mix® 360 in a vertical orientation such that the seeds were approximately half-buried in the soil surface, in 10 cm plastic pots and grown in a (Conviron model BDW-120) at 31° C. during the day, 22° C. during the night, and a 13 hr photoperiod, 220 uE/m². The pots were watered routinely. The survival of the seeds was monitored as well as the emergence of the plantlet shoots through the top of the seed. 48 seeds of each type were planted for this experiment.

The results of the experiment are shown in Table 8. In Table 8, it is shown that the overall survival of the plantlets in the tray based seeds was similar to that observed in the 15 mL centrifuge tubes, but that the square shaped top tray containing seed exhibited a high degree of trapped shoots. In contrast, the conical shaped top tray containing seeds provided low levels of trapping and high levels of emergence.

TABLE 8
Results for the experiment 18 days after planting

| | | Tray - square | Tray -conical |
|---|---|---|---|
| | 15 mL centrifuge | shaped top | shaped top |
| Emerged | 78% | 2% | 80% |
| Dead | 14% | 2% | 2% |
| Trapped | 0% | 94% | 10% |
| Growing but | 8% | 2% | 8% |
| not emerged | | | |
| or trapped | | | |

### Example 12

### Additional Tray Components

In addition to the tray, the seed structure may also contain media. The media includes a solid substrate such as soil, peat, vermiculite, Rockwool, hydrogels, superabsorbents and other materials described in International Publication No. WO 2013/096531, which is included herein by reference. The media also includes water and optionally fertilizers, hormones, antibiotics, antimicrobials and plant growth regulators also described in International Publication No. WO 2013/096531.

The seed structure, either trays, arrays, tube-like structures, or other, may also contain crop chemicals, pesticides, herbicides, fungicides, rodenticides, miticides, nematicides, bird repellants and other chemicals including but not limited to those described in International Publication No. WO 2013/096531. These may be present on the exterior or interior of the enclosure or be included in the media or embedded within the enclosure itself.

### Example 13

### Biodegradable Tray Experiment

Poly(lactic acid) (Ingeo 4032, Natureworks, Minnetonka, Minn.) (70 wt %) was compounded with 30 wt % corn starch (Aldrich, St Louis, Mo.) using a 30 mm twin screw extruder at barrel temperatures of 180° C. The strand was quenched in an ambient temperature water bath and pelletized. The pellets were then dried for approximately 2 days at 50° C. and extruded into approximately 30 mil thick sheet which was thermoformed using a single shuttle machine with an oven temperature of 225° C. and 36-47 second heating duration followed by application of vacuum while using plug assists and 40-80 psig forming pressure. The mold used was a 12 well female mold wherein each well had a rounded square profile (2.5 cm×2.5 cm) at the top with a 4.3 cm long slightly tapered wall down to a 2.2 cm diameter 90 degree included angle conical tip. Each well has multiple holes in the conical tip to allow vacuum to be applied. 6.5 mm holes were punched in the centers of the conical tips of the thermoformed structures. The holes were then widened to 9 mm diameter by the application of a hot air gun to the outside of the shape while applying pressure from the inside using a conical dowel made of polytetrafluoroethylene from the bottom. The trays were then cut apart. These trays were used for the top of the seed structure.

The bottom part of the seed structure was made using PET trays detailed in Example 11.

The PET tray cells were filled with Metromix® 360 soil and sugarcane plantlets that had been regenerated from regenerable tissue for 2 weeks within an array structure in a temporary immersion bioreactor were planted in the soil near the center of the tray cell with their shoots pointing upward. They were then watered to saturate the soil. The poly(lactic acid)/starch tray cell tops were then glued onto the PET bottom tray cells using a hot glue gun in order to create sealed joint. The seeds were placed in another PET tray which held water and were stored/hardened in a growth chamber for 12 days prior to planting. The seeds were then planted in a tilled, furrowed, field, such that the flanges were approximately 1 cm below the soil surface.

After 44 days in the field, the survival of these synthetic seeds was 80%.

### Example 14

### Development of Cellulosic Composites for Sugarcane Artificial Seeds

There are various alternatives for the materials and shapes of the artificial seeds, including the material biodegradability, as described in International Publication No. WO 2013/096531 and International Publication No. WO 2013/096536, both incorporated by reference herein.

Cellulosic pulp was mixed with various materials to study the influence of the materials in the degradation of them in soil. To evaluate the different materials developed with cellulose, an accelerated degradation protocol was developed wherein samples with different compositions were buried in a recipient containing soil, and kept for 1 month in an incubator at 30° C. and approximately 50% humidity, representing field conditions. The cellulose content was measured using thermogravimetric analyses before and after the test to evaluate degradation, indicating cellulose degradation in field.

Different materials were tested, including a residue from the pulp and paper industry containing cellulosic fibers of long and short nature; inorganic fillers, mainly TiO2 and pigments. The final structure contained more than 90% of this cellulose mixture. To this mixture was added polyvinyl alcohol, up to 5% in weight, ionomer of ethylene acid copolymer, up to 5% in weight, used in the packaging industry, cationic starch, up to 5% in weight, more likely 1% to accelerate degradation, while promoting the adherence of the adjacent fibers of cellulose, urea to accelerate the degradation and nutrition of plants up to 10% in weight, more likely 5%, and AKD (alkyl ketene dimer) in a starch solution to serve as moisture barrier by changing the hydrophobicity of cellulose, more likely between 1-10 ppm.

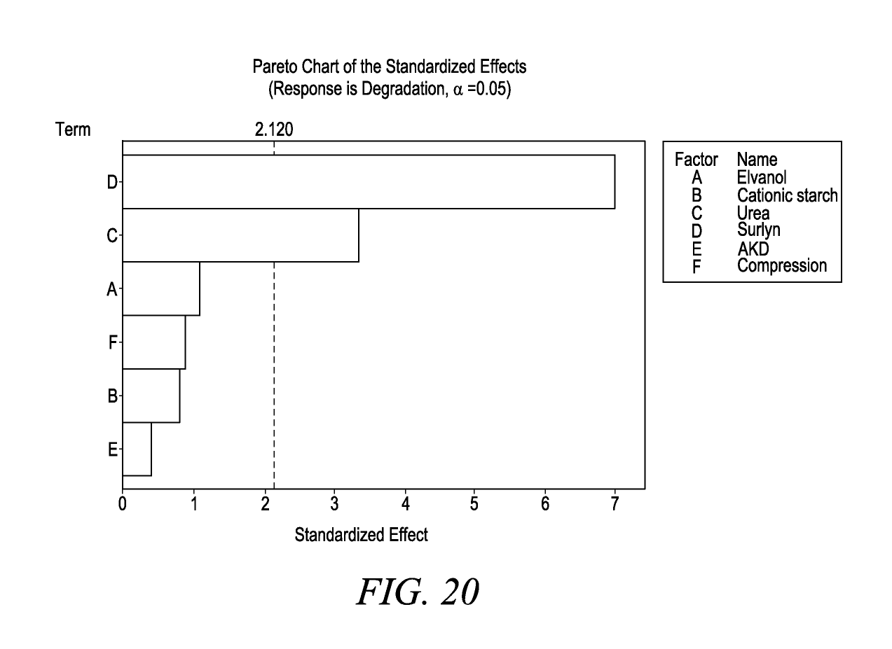

As a result, cationic starch and urea were identified as having a high influence on the degradation of cellulosic compounds as shown in FIG. 20, these results have a confidence interval of 95%. The standardized effect was calculated from the difference between the average degradation response for all samples and the response for each sample with that factor. In FIG. 20, the value 2.120 separates the factors that are significant from those that presented a very low effect.

Several compositions were tested in the field, indicating that the degradation of the structure to allow the plants to establish in soil occurred between 15 and 30 days.

The structure contained cellulosic compounds in the bottom, which is buried in the soil and having a top portion of a clear or translucent polymeric material. The composite material is in contact with wet materials such as saturated substrate, plant, and there may be a need to add an organic coating, biodegradable materials such as -PLA (poly latic acid) or PBS (poly butyl succinate), and their variations. In this example, internal coatings of PLA were tested, but depending on the cultivar, structure geometry and composition, a coating in the outer surface of the composite may be needed.

This example indicated that the interaction among the soil constituents and the cellulosic structures (coated or not) promotes degradation of the cellulose in a rapid rate, even with the protective coatings.

### Example 15

### Seed Assembly Process

In this example, the process for assembling the seeds is a multistep process. The first step utilizes equipment to handle the tray, fill it with soil, and prepare the tray for the plant. The next step involves transplanting the plantlet from the Stage 3 bioreactor to the tray. This can be done in several ways, depending on how the plantlets are grown in the bioreactor. For plantlets that are grown in an array, transplanters can be used to pluck the plantlet from the array and insert it into the tray. Or the array and tray can be aligned vertically and the plantlet can be pushed through the array and into the tray. Both methods are relatively quick and can be accomplished with low cost equipment. If the plantlets are grown as a high density mat or sod, then a single plantlet or small cluster of plantlets can be grabbed and inserted into the tray (similar to how a rice transplanter operates).

Once the trays have been fully populated with plantlets a cover tray is placed onto the bottom tray. One may thermally bond the top and bottom trays together. One may use RF welding since this is inexpensive and the entire tray can be bonded at once in a few seconds. However, many materials are not RF weldable so either an RF susceptor is blended into the polymer or an alternate method like a hot bar, ultrasonic welding, or adhesive is used. It is possible to design the trays so that they snap together so that no bonding method is needed at all. Once the trays are fully assembled they are prepared for storage. This may involve loading multiple trays into a shipping carton or placing individual trays in a plastic sleeve and shrinking the sleeve onto the trays to provide added protection and stiffness.

### Example 16

### Artificial Seeds—Tube-like Structures

### Meristem Isolation and Culturing

Sugarcane stalks (approximately 6-9 months old) were grown in the field or greenhouse such that the apical meristem was at least 30 cm above the soil line. Stalks were cut near the base of the stalk. Leaf blades were trimmed leaving the leaf sheaths intact. Stalks were cut into 20 cm long pieces with the apical meristem in the center. One to three outer leaf sheaths were removed and the stalk pieces were submerged in 70% ethanol for 20 minutes. The stalks were moved to a clean container inside a laminar flow hood. A water rinse is optional.

Successive leaf sheaths were removed to expose the meristem. The stalk pieces were further trimmed leaving 1-2 cm margins around the meristems. The meristem section was split in half longitudinally with the cut either separating or bisecting the axillary buds. The two halves were placed cut side down onto proliferation agar media (0.8% agar or 0.43% phytagel containing full strength Murashige-Skoog salts+vitamins (MS) (Phytotech Labs, Overland Park, Kans.)+3% sucrose and 4 mM BAP (6-Benzylaminopurine); 0.8% agar; or 0.43% Phytagel (Sigma-Aldrich, St. Louis, Mo.), in disposable plastic Petri dishes.

Material was incubated at 26° C. in growth chambers or rooms with approximately 30 uE/m2/s of broad spectrum light intensity (PAR). Each ‘half-explant’ was trimmed at 7-10 days and placed onto fresh media, this step was repeated 3 times for a total growth time of 28 days on agar media. Proliferating meristems, also referred to as mericulture tissue, were trimmed and placed into temporary immersion reactors to generate regenerable plant tissue.

### Culturing of Regenerable Plant Tissue

Half-explants were trimmed of excess stalk tissue, leaving the proliferating buds areas. Trimmed tissue was placed into a temporary immersion chamber for liquid culture. The reactor was placed in a growth chamber, or room, with 30 uE/m2/sec of PAR light, and was attached to tubing connected to pumps to allow standard immersion cycling. Media (MS+3% sucrose+4 mM BAP+3 mg/L paclobutrazol) was changed weekly. Harvest occurred 4 weeks after initiation. The mericultured buds were loosely fragmented, segregated into 4 g batches and placed into a second round of temporary immersion for an additional 3 weeks.

### Plantlet Formation

Plantlet formation (production of plants with shoots and roots), also called regeneration, was initiated by transferring the proliferating plant material to media without hormones. Fragments were made using scalpels. Fragment size for growth in arrays or in 1 liter temporary immersion reactors were approximately 1-10 mm per side cubes. Fragments were loaded by hand using aseptic technique into an array temporary immersion reactor. The media used was MS+3% sucrose. Plantlet formation occurred at 26° C. in 60 uE/m2/sec of PAR light (16 hours on and 8 hours off), and media was cycled over the fragments for 1 minute every 2 hours. Media was replaced weekly and was supplemented with 0.2% PPM (Plant Preservative Mixture™, Plant Cell Technology Inc. Washington, D.C.), and/or 100 ppm cefotaxime as needed. Supplemental carbon dioxide (1500 ppm) was used in some growth cycles, but is not essential. For weeks 3-4, the MS concentration was decreased to ½ strength to encourage rooting. The sucrose levels were also decreased to 2% then 1% during this time. Arrays were harvested after 14 days and plants removed by hand or using multi hole punch system to be placed into structures for hardening and field planting.

### Seed Assembly and Hardening

The seed was assembled by placing a staple in the end of a tube-like structure (0.4″ diameter, 9″ length, clear) and filling the tube-like structure with 6″ of MetroMix® 360 potting media. One hundred tube-like structure were filled with soil and placed in a 1 L beaker of water to saturate the substrate. Once the substrate was wet, one plant which was regenerated for 2 weeks, was placed on top of the substrate. The assembled seeds were placed in a 1 L beaker and water was added to the 200 mL line. The beaker was placed in a screen house for 14 days. The water level was maintained throughout the duration of 14 days. Ninety-nine percent of the seed survived during the 2 week hardening stage.

### Seed Evaluation

After 14 days in the screen house, the plants were planted in the soil at a depth of 6″. After 70 days, the survival rate of the plants was evaluated. The tube-like structure-based seeds had a survival rate of 60%.

### Example 17

### Regeneration of Plants from Micropropagated Fragments Directly into Short, Tube-Like Structures

### Short, Tube-Like Structures

Regeneration of vigorous, hardened and well rooted sugarcane plants from micropropagated fragments (2 mm to 5 mm) that includes a solid substrate (sieved potting soil, vermiculite or similar) may be performed in short, tube-like structures, which allows for improved development of the root system and minimizes the risk of damage upon subsequent handling. Duration of the in vitro sterile condition phase for plant regeneration is reduced to 7 days or, optionally, 14 days contrasted to 4 to 5 weeks of conventional plant regeneration methods. Therefore, the short tube-like structures allow the in vitro plantlets to be transferred to the greenhouse at 7 or 14 days of regeneration, and at two additional weeks in the greenhouse, they are ready for encapsulation in a structure designed for planting in the field, with minimal damage to the root system. The small tube-like structures also simplify the development of an automated handling of the plant units during the various processing steps until encapsulation. The original small tubes with developed plants can be transferred directly to small pots or larger tubes, without encapsulation, and immediately moved to a shadehouse or open nurseries for further growing before transfer to the field.

Short tube-like structures are made of, but not limited to, plastic (e.g. PP, PET, PVC) or of biodegradable material (e.g. paper, cellulose, waxy) stabilized at the bottom of a temporary immersion system (TIS) bioreactor or in any other container under sterile or non-sterile condition, and can be incubated in a growth chamber or in the greenhouse.

### In Vitro and Greenhouse Experiments

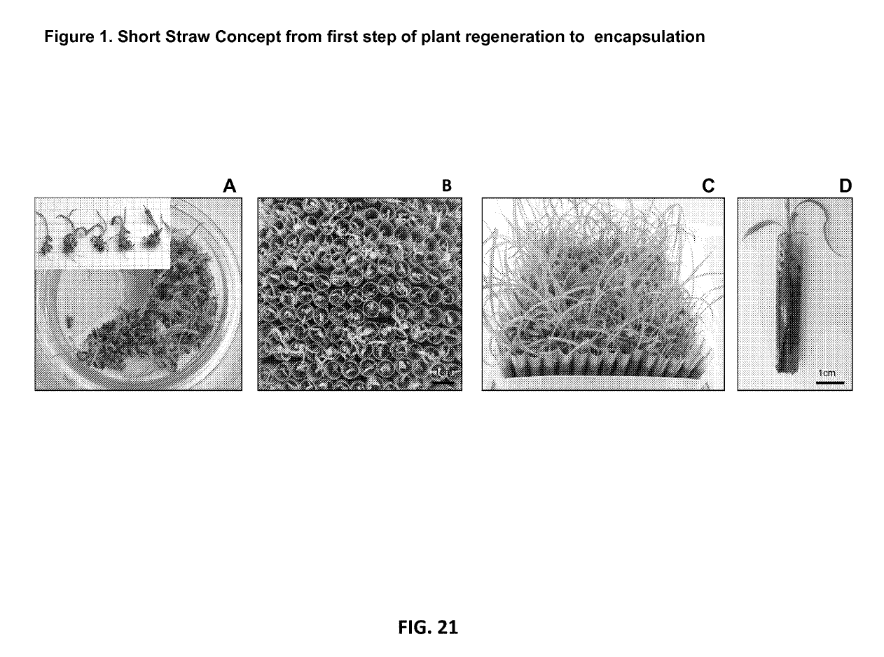

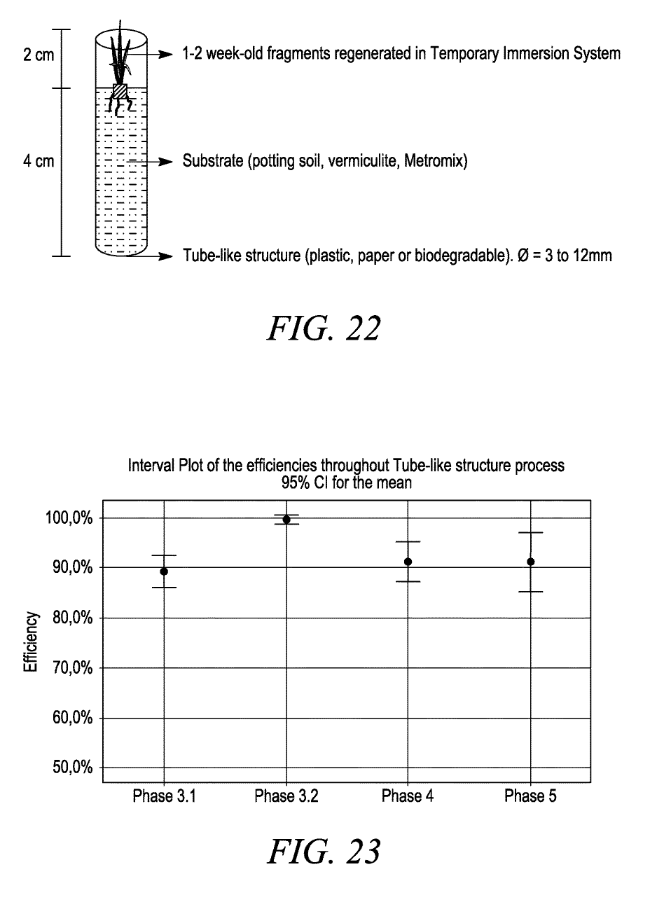

The in vitro micropropagation cultures at Phases 1 and 2 followed the protocol for sugarcane described herein (see previous examples). The proliferating meristematic tissues cultured into a RITA temporary immersion system (TIS) during 4 weeks (Phase 2.1), or for additional 4 weeks (phase 2.2), were sliced into 2 mm to 5 mm diameter fragments. Plant regeneration (Phase 3) of the fragments was initiated by 7-day incubation in a RITA (Phase 3.1) containing liquid culture medium comprising MS salts, MS vitamins, sucrose and, optionally, supplemented with activated charcoal (FIG. 21A). Cultures were maintained in a growth chamber adjusted to 26° C., 30 μE m⁻² s⁻¹ PAR, and 14/10 h light/dark photoperiod. The regenerating fragments (FIG. 21B) were transferred onto solid substrate (sieved potting soil, vermiculite) contained in a tube-like structure (3 to 12 mm diameter×40 to 70 mm of length) made of biodegradable waxed paper or non-biodegradable material tube-like structure, such as a straw (FIG. 22). The tubes were previously stabilized vertically onto a plastic grid placed at the bottom of the culturing container. These 7-day regenerating cultures, now placed onto solid substrate in the small tube-like structures, were either transferred (a) to a larger bioreactor (PlantForm) for another TIS cycle of 7 days (Phase 3.2; FIG. 21B) with the liquid culture medium without charcoal, under 60 μE m⁻² s⁻¹ in the growth chamber, before being removed from aseptic condition and transferred to the greenhouse or (b) directly to the greenhouse under non-aseptic condition (Phase 4; FIG. 21C). Upon removal from aseptic in vitro conditions, in both procedures the substrate was rinsed with filtered water in order to remove sucrose. Cultures were then transferred to the greenhouse in an open tray in order to advance plant development and hardening with the tubes partially immersed into nutrient solution (FIG. 21C). After 2 to 3 weeks the well developed and hardened plants were encapsulated in a structure designed to direct transfer to the field, without being removed from the original tube-like structure (FIG. 21D), thus preserving the root system. Otherwise, they were transferred to the screenhouse for further development without encapsulation.

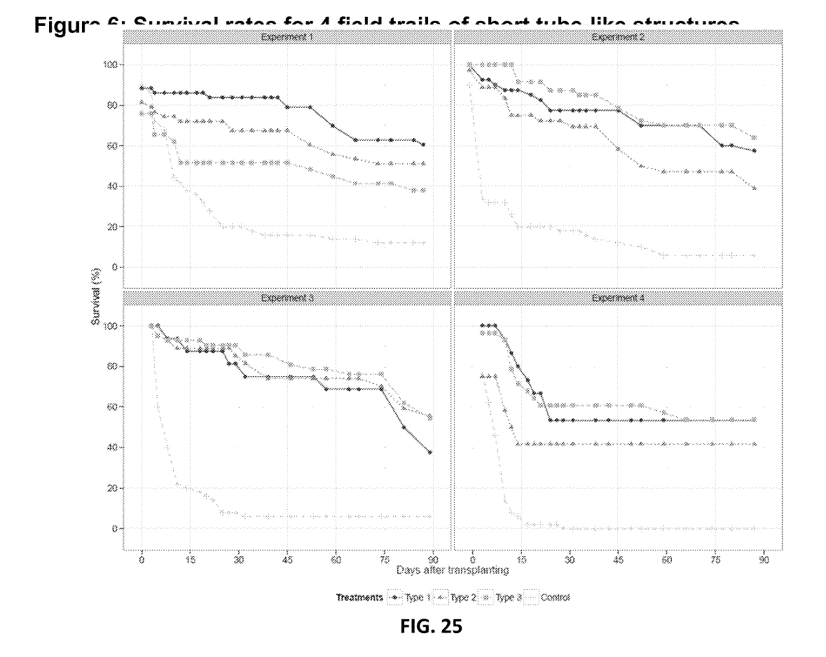

Results from experiments demonstrating the short, tube-like structures are shown in FIGS. 21A-D-FIG. 24. The field trial data is summarized in FIG. 25.

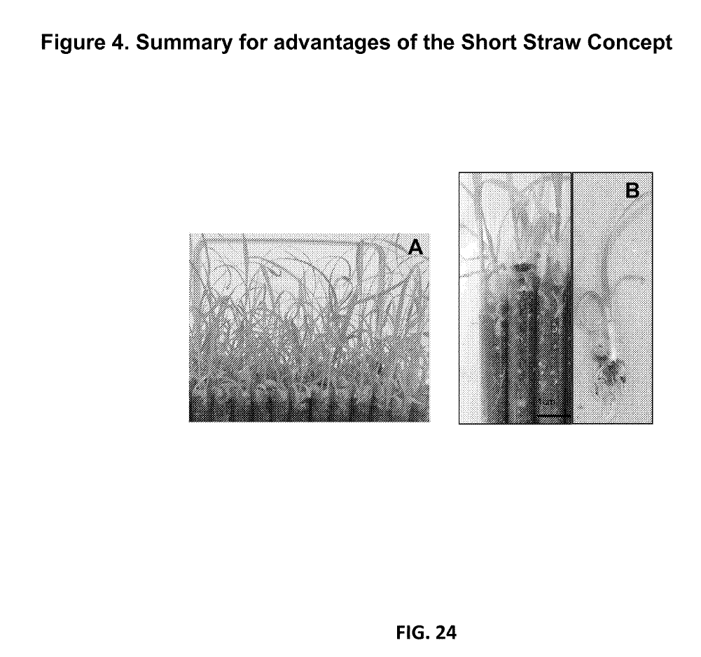

Up to 11 experiments for each phase of the experiment for tube-like structures were carried out in the growth chamber followed by the greenhouse. The data of plant recovery and survival from these experiments are summarized in FIG. 23. The average survival rate of the fragments regenerated during 7 days in TIS in vitro condition averaged about 90% and did not diverge much from plants that remained 14 days TIS. By allowing these plantlets to grow in the greenhouse for additional 2 or 3 weeks, they were grown into plants that were mostly undistinguishable from one another at 28 days of regeneration. The reduced time length of the plants cultured for only one week in the in vitro TIS did not affect plant quality (FIG. 24).

Field Survival with Tube-Like Structures

Tube-like structures, made of plastic (6 cm long, 8 mm in diameter) and filled with sieved potting soil as substrate. Three variations of the handling of the tube-like structures were tested:
Type 1: Plant fragments for one week in temporary immersion reactors (aseptic condition with liquid media including sugar and nutrients), transplanting of one plantlet to each tube-like structure with potting soil, another week in temporary immersion (aseptic condition with liquid media including sugar and nutrients), transfer of the reactor to the greenhouse for two weeks (water or liquid media with nutrients only and under continuous immersion). Perform two lateral cuts on the tube-like structure before transplanting into another structure. Keep the structure for other two weeks in the greenhouse before transplanting to the field.
Type 2: Identical to type 1, except for the complete removal of the tube-like structure before transplanting to the structure.
Type 3: Plant fragments for one week in temporary immersion reactors (aseptic condition with liquid media including sugar and nutrients), transplanting of one plantlet to each tube-like structure with potting soil, transfer of the reactor to the greenhouse for three weeks (water or liquid media with nutrients only and under continuous immersion). Perform two lateral cuts on the tube-like structure before transplanting into another structure. Keep the structure for other two weeks in the greenhouse before transplanting to the field.

For the control, treatment plants were obtained from fragments maintained in temporary immersion for 4 weeks before transplanting to the structure. The plants in the structures were kept for two weeks in the greenhouse before transplanting to the field.

The basic form of the structure used for planting the plants in the field was an octahedron, 10 cm high and a 2.5 cm square base. The bottom half of the structure was filled with a peat moss like substrate. Holes existed at the top and the bottom of the structure. The hole on the top was open and closed with Parafilm® on the bottom.

The greenhouse used for keeping the plants before transplanting to the field had some means for avoiding excessive temperatures: a cooling pad and ventilators. Ventilators were activated whenever temperatures reached 25° C.

The experiment was repeated four times with 50 replicates per treatment. Survival three months after transplanting to the field ranged between 40 and 60% for all treatments using a tube-like structure during regeneration (FIG. 25). These results represent an improved survival rate compared to the control treatment, which was typically below 10%. No systematic ranking of the different treatments with the tube-like structures emerged when comparing the four experiments.

### Example 18

### Seed Planting Process

The seed structures, for example as described in Example 11, are delivered to a sugarcane planter in a cartridge with the seeds oriented in the cartridge. The planter feeds the cartridges with the seed trays into the seed delivery zone, actuators singulate the seeds and move them to the planting zone. The planter places the seeds vertically at the appropriate spacing and depth and firms the soil around the seed leaving the top part uncovered.

The planter creates an environment that is conducive to optimal seed survival. In minimum tillage conditions, the planter prepares a clean row and conditions the soil so that the seed can be placed into a good seedbed. In dry soils, the planter creates a shallow furrow in order to plant the seed in a moister soil regime, thereby improving chances of survival.

The planting equipment may also puncture the individual seeds to provide an escape route for the roots and the shoots.

### Example 19

### Micropropagation of Genetically Modified Sugarcane

Meristems from 15 genetically modified sugarcane plants and 15 non-genetically modified sugarcane plants were micropropagated using the process outlined in International Publication No. WO 2013/096531, with a few modifications. For the work in this example, during stage 1.0, freshly excised tissue of both genetically modified and non-genetically modified was placed directly onto the proliferation agar medium without undergoing the alternative process of bleach sterilization. After 4 weeks in stage 1.0 and 4 weeks in stage 2.1, the developing bud tissue was fragmented and transferred to regeneration in temporary immersion RITA vessels containing wide mesh inserts. Initiation of regeneration was done with 200 mls of full strength MS+3% sucrose, no hormones and the addition of 100 ppm of cefotaxime for prophylactic control of contamination. Vessels were transferred to an Adaptis growth chamber to allow both genetically modified and non-genetically modified to grow for the full 4 weeks of regeneration.

After 4 weeks in regeneration, both genetically modified and non-genetically modified were singulated into plantlets and 100 plantlets per genetically modified and non-genetically modified were transferred into separate flats containing Metromix® 360 and grown for 33 days in a controlled environment with daily watering. After 33 days, all plantlets were transferred into 4-inch pots filled with Metromix® 360 with 2-4 plantlets per pot. At the time of planting in the greenhouse, the genetically modified and the non-genetically modified plants were comparable in plant health.

70 genetically modified plants in 23 pots and 69 non-genetically modified plants in 22 pots were generated by this process after transferring them to soil (when the dewlaps of the plants were about 12-inches from the soil). All plantlets were grown in a greenhouse environment.

It is concluded that this sugarcane micropropagation process does not impact the expression of the transgene in the genetically modified sugarcane.

That which is claimed:

1. A method of planting sugarcane, said method comprising:
(i) obtaining a regenerable plant tissue comprising,
a. growing excised sugarcane meristem tissue on solid support media in the presence of plant hormones, and
b. transferring the tissue from step (a) to liquid growth media in the presence of plant growth regulators; and growing to form the regenerable plant tissue;
(ii) subdividing said regenerable plant tissue comprising,
a. cutting said tissue into smaller segments having a size from about 1 mm³ up to about 1000 mm³,
b. placing said cut tissue into temporary immersion with liquid medium for at least about 3-10 days, and
c. replacing the medium after about every 3-10 days;
(iii) further growing said subdivided regenerable plant tissue, comprising,
a. placing the plant tissue into at least one of the following:
i. a cavity of an array structure,
ii. a tube-like structure, or
iii. a support structure; and
b. growing said tissue;
(iv) forming plantlets from said regenerable plant tissue by growing in media lacking hormones;
(v) transferring the plantlets from the structures of (iii) to an artificial seed;
(vi) forming plants from said plantlets, comprising exposing the plantlets to one or more of the following conditions:
a. increased light intensity,
b. reduced humidity, and
c. contact of plant roots with media supports such as Rockwool, Metro Mix 360®, soil or other material;
(vii) transporting said artificial seed structure with said plants, comprising,
a. introducing said artificial seed structure into a space, and
b. transporting said seed structures to a field; and
(viii) planting the artificial seed structure.

2. A method of planting sugarcane, said method comprising:
(i) obtaining a regenerable plant tissue comprising,
a. growing excised sugarcane meristem tissue on solid support media in the presence of plant hormones, and
b. transferring the tissue from step (a) to liquid growth media in the presence of plant growth regulators; and growing to form the regenerable plant tissue;
(ii) subdividing said regenerable plant tissue comprising,
a. cutting said tissue into smaller segments having a size from about 1 mm³ up to about 1000 mm³,
b. placing said cut tissue into temporary immersion with liquid medium for at least about 3-10 days, and
c. replacing the medium after about every 3-10 days;
(iii) further growing said subdivided regenerable plant tissue in an artificial seed structure;
(iv) forming plantlets from said regenerable plant tissue by growing in media lacking hormones;
(v) forming plants from said plantlets, comprising exposing the plantlets to one or more of the following conditions:
a. increased light intensity,
b. reduced humidity, and
c. contact of plant roots with media supports such as Rockwool, Metro Mix 360®, soil or other material;
(vi) transporting said artificial seed structure with said plants, comprising,
a. introducing an artificial seed structure into a space, and
b. transporting said seed structures to a field; and
(vii) planting the artificial seed structure.

3. The method of claims 1 and 2, wherein step (i) (a) is repeated at least once.

4. The method of claims 1 and 2, wherein said regenerable plant tissue in step (ii) is subdivided using at least one of the following: chopping, automated slicing, automated grinding, or sieving.

5. The method of claims 1 and 2, wherein there is at least a 100-×-fold amplification of plantlets for each meristem excised.

6. The method of claims 1 and 2, wherein the media further comprises compounds selected from the group consisting of: antibiotics, fungicides, plant hormones, growth nutrients, salts, plant hormone inhibitors, growth regulators, agars, and insoluble materials.

7. The method of claims 1 and 2, wherein the media further comprises endophytes.

8. The method of claim 1, wherein the array comprises a multi-cavity support for tissue growth used in continuous or temporary immersion tissue culture.

9. The method of claim 1, wherein the array cavities are circular, square, rectangular, or multishaped.

10. The method of claim 1, wherein the array cavities are formed by machining, molding or any other suitable means such as welded honeycomb structures; and have a cavity diameter of about 1/16 to ½ inch.

11. The method of claim 1, wherein the regenerable plant tissue is placed in said array cavities by at least one of the following: manual placement, vibration distribution, vacuum using a transfer plate with suction patterns matching the cavity array pattern, distribution while floating in growth media, suspension in liquid followed by injection, or by grating, smashing or rollering tissue into array cavities.

12. The method of claims 1 and 2, further wherein said array cavity or artificial seed structure has an upper and lower section separated by a tissue fragment support, the fragment support positioning the fragment vertically within the cavity creating shoot and root growth zones, wherein said shoot and root growth zones are of adequate thickness to allow plantlets to grow without becoming entwined with plantlets in adjacent cavities.

13. The method of claim 1, wherein inserting the plantlet into the artificial seed structure comprises transferring said plantlet from an array structure to a tray structure using a method selected from the group consisting of piston force transfer, vacuum transfer, sieving, manual transfer, and mechanical transfer.

14. The method of claims 1 and 2, wherein said artificial seed structure is stored in conditions comprising:
a. maintaining a moisture content within said seed structure of between 40%-80%,
b. supplying a light source, and
c. maintaining a temperature of at least 10 C and 30 C
wherein the fully formed plants remain viable for at least one week.

15. The method of claims 1 and 2, wherein the artificial seed structure comprises compounds selected from the group consisting of: soil, other rooting media, fungicides, nematicides, antibiotics, insecticides, antimicrobials, biocides, herbicides, plant growth regulator or stimulator, endophytes, mollucicides, miticides, acaricides, bird repellants, insect repellants, rodent repellants, growth nutrients, salts, plant hormone inhibitors, agar, and insoluble materials.

16. The methods of claims 1 and 2, further wherein the excised meristems, the regenerable tissue, and the plants are genetically modified.
